(19) United States
(12) Patent Application Publication
(10) Pub. No.: US 2005/0014680 A1
(43) Pub. Date: Jan. 20, 2005

(54) Methods and compositions for enhancing neuron growth and survival

(76) Inventors: Gerald R. Crabtree, Palo Alto, CA (US); Isabella Graef, Palo Alto, CA (US); Marc Tessier Lavigne, Palo Alto, CA (US)

Correspondence Address: ROPES & GRAY LLP, ONE INTERNATIONAL PLACE, BOSTON, MA, 02110-2624, US

(21) Appl. No.: 10/824,939

(22) Filed: Apr. 15, 2004

Related U.S. Application Data
(60) Provisional application No. 60/462,909, filed on Apr. 15, 2003.
(60) Provisional application No. 60/474,546, filed on May 30, 2003.

(51) Int. Cl. A61K 38/28; A61K 38/18

(52) U.S. Cl. 514/3; 514/12

(57) ABSTRACT

Pharmaceutical compositions of NF-AT agonists may be used to promote nerve regeneration or to reduce or inhibit secondary nerve degeneration which may otherwise follow primary CNS or PNS injury, e.g., trauma (e.g., blunt trauma, penetrating trauma), compression [e.g., compression due to tendons and/or inflamed synovial membrane such as in carpal tunnel syndrome], bones [for instance sciatica], or growths [benign or cancerous, including growth of the nerves themselves or of surrounding tissue]) hemorrhagic stroke, ischemic stroke or damages caused by surgery such as tumor excision. In certain embodiments, NF-AT agonists may be used to treat spinal cord injuries and promote nerve grafts.

### RELATED APPLICATIONS

This application claims priority to U.S. Provisional Application Nos. 60/462,909 and 60/474,546, filed Apr. 15, 2003 and May 30, 2003, respectively, both entitled “Methods and Compositions for Enhancing Neuron Growth and Survival.” The entire teachings of the referenced applications are incorporated by reference herein.

### STATEMENT REGARDING FEDERAL FUNDING

Certain work described herein was funded in part by grants from the National Institute of Health. The United States government has certain rights in this invention.

### BACKGROUND OF THE INVENTION

The nervous system includes the CNS and the PNS. The CNS is composed of the brain and spinal cord; the PNS consists of all of the other neural elements, namely the nerves and ganglia outside of the brain and spinal cord.

Damage to the nervous system may result from a traumatic injury, such as penetrating trauma or blunt trauma, or a disease or disorder, including but not limited to Alzheimer's disease, Parkinson's disease, multiple sclerosis, Huntington's disease, amyotrophic lateral sclerosis (ALS), diabetic neuropathy, senile dementia, and ischemia.

The complex yet stereotyped morphologies of neurons arise during embryonic development through the growth of axons and dendrites from neuronal cell bodies. Extrinsic and intrinsic factors both contribute to shaping these extensions (Edlund and Jessell, 1999; Gao et al., 1999). A variety of extracellular cues, including such molecules as the netrins and neurotrophins, stimulate, inhibit, and guide process extension and branching by binding receptors present on axonal and dendritic growth cones and along the axonal and dendritic shafts (Giger and Kolodkin, 2001; Huang and Reichardt, 2001; Tessier-Lavigne and Goodman, 1996). At the same time, how the neuritic processes respond to these cues is determined by developmental programs within neurons, which dictate the nature of the receptors for extracellular cues and the signal transduction molecules that are active.

There is mounting evidence for the existence of dedicated transcriptional programs, acting after the initial specification of neurons into generic classes, that regulate later aspects of their development including the choice of pathway made by their axons or the shape of their dendritic arbor. In the case of spinal motorneurons, for example, initial specification of motorneurons occurs through the action of particular homeodomain and basic-helix-loop-helix transcription factors (Anderson, 2001; Briscoe et al., 2000; Shirasaki and Pfaff, 2002). However, their subsequent choice of major axonal pathways (correlating with their columnar identity in the spinal cord) is directed by distinct combinations of LIM homeodomain transcription factors (Kania et al., 2000; Sharma et al., 1998; Thor et al., 1999; Tsuchida et al., 1994). Another example of transcription factors regulating later aspects of neuronal morphogenesis is provided by the homeodomain transcription factor Otx1, which is required for the regulation of stereotyped pruning of layer 5 cortical neuron branches (Weimann et al., 1999). Furthermore, genetic screens in Drosophila have identified the zinc finger protein sequoia, as an important regulator of dendrite development (Brenman et al., 2001; Gao et al., 1999) and the zinc finger protein brakeless, as a gene critical for axon targeting during visual system development (Rao et al., 2000; Senti et al., 2000). Although some of these transcriptional processes may be activated autonomously in neurons simply as a consequence of an early specification event, in other cases their action may be regulated by 1 ate environmental signals, allowing, for example, for fine-tuning of the timing of their activation. Expression of various ETS family transcription factors, which appear to control late aspects of neuronal morphogenesis (Arber et al., 2000), are regulated by the contacts of neurons with their targets (Lin et al., 1998). The picture that is emerging, therefore, is that specific aspects of neuronal morphogenesis may be controlled by dedicated transcriptional programs, some of which may be regulated by environmental cues. However, the range of neuronal properties that are controlled by changes in gene expression and the identity of key transcriptional regulators of such events, remain largely unknown.

### BRIEF DESCRIPTION OF THE INVENTION

The invention discloses methods for promoting axonal growth comprising treating a neuron with an NF-AT agonist. In one embodiment, the invention discloses the use of two or more NF-AT agonists in the above described method.

In one embodiment, the NF-AT agonist interacts with NF-AT and modulates its nuclear translocation. In another embodiment, the NF-AT agonist binds NF-AT and increases its nuclear localization. In another embodiment, the NF-AT agonist interacts with calcineurin and increases the dephosphorylation of NF-AT.

In one embodiment, the NF-AT agonist is calcineurin or an agent that upregulates the expression of calcineurin. In another embodiment, the NFAT agonist interacts with calmodulin and increases the activity of calcineurin and the dephosphorylation and/or activation of NF-AT. In another embodiment, the NFAT agonist stimulates an increase in intracellular calcium concentration which induces the activation of calcineurin.

In another embodiment, the NF-AT agonist is an inhibitor of GSK3. In one embodiment, the inhibitor of GSK 3 is selected from the group consisting of an RNAi molecule, a ribozyme or a DNA enzyme that inhibits the expression of GSK3.

In another embodiment, the NF-AT agonist modifies the DNA interaction of NF-AT in order to increase NF-AT dependent transcription.

In another embodiment, the NF-AT agonist modifies the interaction of NF-AT with a nuclear partner that results in an increase in transcription.

In another embodiment, the NF-AT agonist increases or enhances the expression of NF-AT. In another embodiment, the NF-AT agonist increases or enhances the expression of NF-ATc4.

In one embodiment, the NF-AT agonist is a netrin. In another embodiment, the NF-AT agonist is a neurotrophin. In another embodiment, the NF-AT agonist is not a netrin or a neurotrophin.

The invention further comprises a method to activate NF-AT dependent gene transcription comprising the use of a netrin or a neurotrophin.

The invention further comprises a method for promoting axonal growth comprising treating a neuron with a composition comprising an NF-AT agonist and another agent selected from the group consisting of: a neurotrophic factor, a neuropoietic factor, inosine, a fibroblast growth factor, an insulin-like growth factor, a platelet-derive growth factor, an anti-inflammatory, anti-NGF, anti-BDNF, anti-IGF-I, transforming growth factor-beta 1, other agents that increase production of inducible-nitric oxide synthase (i-NOS), an activator of macrophages, LPS, indomethacin, and a leukemia inhibitory factor (LIF).

The invention also comprises a method for promoting axonal growth comprising administering an NF-AT agonist in a biodegradable nerve conduit.

The invention further comprises a method to induce regeneration of neurons comprising treating said neurons with an NF-AT agonist.

The invention also relates to a pharmaceutical composition comprising and NFAT agonist and a pharmaceutically acceptable carrier.

The invention also relates to methods of identifying compounds that are NF-AT agonist and promote axonal growth.

In one embodiment the method of identifying such compounds comprises: (a) contacting the compound with a cell comprising NF-AT; (b) determining the location of NF-AT within the cell in the presence and in the absence of the compound, wherein an increase of NF-AT in the nucleus indicates that the compound is an NF-AT agonist; and (c) determining whether the compound promotes axonal growth. Such determination can be made in any model system for axonal regeneration.

In another embodiment, the method of method of identifying a compound that is an NF-AT agonist and promotes axonal growth comprises: (a) contacting a cell expressing NF-AT with a compound; (b) determining the phosphorylation state of NF-AT in the presence and absence of the compound; wherein a decrease in the phosphorylation of NF-AT indicates that the compound is an NF-AT agonist; and (c) determining whether the compound promotes axonal growth.

In another embodiment, the method of identifying a compound that is an NF-AT agonist and promotes axonal growth comprises: (a) contacting NF-AT with a phosphatase under conditions that allow the dephosphorylation of NF-AT in the presence and in the absence of a compound, (b) determining the phosphorylation state of NF-AT, wherein an decrease in the phosphorylation indicates that the compound is an NF-AT agonist; and (c) further determining whether the compound promotes axonal growth. In one embodiment, the phosphatase is calcineurin.

In another embodiment, the method of identifying a compound that is an NF-AT agonist and promotes axonal growth comprises: (a) contacting NF-AT with a kinase under conditions that allow the phosphorylation of NF-AT in the presence and in the absence of a compound, (b) determining the phosphorylation state of NF-AT, wherein an decrease in the phosphorylation indicates that the compound is an NF-AT agonist; and (c) further determining whether the compound promotes axonal growth. In one embodiment, the kinase is GSK-3.

In another embodiment, the method of identifying a compound that is an NF-AT agonist and promotes axonal growth comprises: (a) transfecting a cell with an expression vector comprising a nucleic acid encoding a reporter gene operatively linked to an NF-AT dependent transcriptional regulatory sequence; (b) incubating the cell in the presence and absence of a compound; (c) measuring the expression of the reporter gene; wherein an increase in the expression of the reporter gene indicates that the compound is an NF-AT agonist; and (d) further determining whether the compound promotes axonal growth in a model system for axonal regeneration.

### BRIEF DESCRIPTION OF THE FIGURES

FIG. 1: Axon Guidance Defects in NFATc2/2/4 Mutant Embryos.

(A-F) Whole-mount immunostaining with anti-neurofilament (NFM) antibody on E10.5 wild-type (A, C, and E) and NFATc mutant (B, D, and F) embryos.

(C-D) The three branches of the trigeminal ganglion (V), (oph, ophthalmic, max, maxillary; and mand, mandibular) fail to extend in NFATc mutant embryos. Arrow in (D) shows descending tracts at the Vth nerve root. HB, hindbrain; rostral is up.

(E-F) Peripheral projections from the dorsal root ganglion (DRG, arrowhead) are severely shortened in the NFATc mutants (F). In mutant embryos (F), sensory afferents from the DRG to the spinal cord (arrow) fail to extend longitudinal tracts alongside the spinal cord. DREZ-dorsal root entry zone (arrow in E) DF-dorsal funiculus dorsal is up.

(G-H) Transverse sections of E10.5 wild-type. (G) and mutant (H) embryos stained with anti-NFM antibody. Dorsal is up. Mutant embryos display ventromedially directed projections (arrow in H).

(I-J) Projections of spinal commissural axons (arrowhead in I) to the floor plate (FP), where they cross the midline (open arrowhead), are seen in the control littermates (I). In mutant embryos (J), no commissural axons reach the floor plate. The position of commissural neuron cell bodies is indicated by the asterisk in (J). Some TAG-1 positive processes grow dorsally along the edge of the spinal cord (arrow in J). Scale bar for (A-B) is 1.5 mm; (C-F) is 600 μm; and (G-J) is 50 μm. The difference in intensity of the staining between wt and mutant embryos reflects the higher NFM expression in the mutant embryos and does not represent a difference in exposure.

FIG. 2: illustrates that pharmacological calcineurin inhibition during embryonic development produces defects similar to those in NFATc2/c3/c4 Mutant embryos.

(A-F) Whole-mount anti-NFM staining at E10.5 shows sensory axon projection abnormalities in embryos from mothers treated with 25 mg/kg CsA twice per day on E7.5 and E8.5.

(A-D) Trigeminal ganglia; rostral is up. Peripheral trigeminal processes are shortened in the CsA-treated embryos (low magnification in B, higher magnification in D) and show few thin peripheral axons (arrow in D).

(E, F) Dorsal root ganglia; dorsal is up. Peripheral axons from the dorsal root ganglion (arrowhead) fail to extend in CsA treated embryos (F). CsA treated embryos also show a failure of formation of the dorsal funiculus seen in the nontreated age-matched control (arrow in E).

(G) Maternal administration of CsA for 3 hours on E10.5 (lane1) induced phosphorylation of the NFATc4 protein compared to non-treated embryos (lane2). NFATc4 is dephosphorylated in E11.5 trigeminal ganglia (lane 3). The mobility of NFATc4 in E13.5 DRG, cortex, spinal cord indicates that it is dephosphorylated and hence active. The E13.5 liver or yolk sac contains little or no NFATc4 while the heart shows a prominent phosphorylated band. The band indicated by the asterisk (*) is a cross reacting band and does not represent an NFATc4 isoform, since it is present in wt and mutant embryos. Protein loading is assessed by probing the blots with an anti-HSP-90 antibody (G, bottom panel).

Scale bar for (A,B) and (E,F) is 100 μm and for (C) through (D) is 50 μm.

FIG. 3: illustrates the cell autonomous nature of the defect of sensory axon growth.

(A-C) E10.5 trigeminal explants were grown for 48 hours in a three dimensional collagen matrix. Axonal outgrowth was visualized by staining with anti-NF-M antibody. Whereas wild-type ganglia showed robust outgrowth (A), NFATc triple mutant ganglia failed to extend axons in vitro (B). Wild-type explants treated with FK506/CsA at the onset of the culture period (C) also showed an absence of axonal extension.

(D-I) Dissociated trigeminal neurons cultured on laminin and stained with anti-NF-M antibody (red) and DAPI (blue). (D and G) littermate control; (E and H) NFATc mutant cells; (F and I) cells treated for 24 hr with FK/CsA.

(J-O) Trigeminal explants were cultured on matrigel and axons were visualized by anti-NF-M staining. Neither mutation of NFATc2/c3/c4 (K) nor treatment of explants with FK506/CsA (L) in the presence of NT-3 and NGF affected axon elongation in matrigel. Explants cultured in the absence of NT-3 and NGF also showed no impairment of neurite growth in matrigel (M-O).

Scale bar for (A) is 500 μm, (B-C) 250 μm, (D-F) 30 μm, (G-I) 10 μm; and (J-O) 650 μm.

FIG. 4: Neither Calcineurin nor NFAT/c2/c3/c4 Are Required for Sensory Neuron Survival in Vivo or in Vitro.

(A-D) Tunel (green) and nuclear (DAPI, blue) stain of E10.5 transverse sections; dorsal is up. Tunel-positive cells are indicated by arrow in (A), (NT)-neural tube.

(E) Bars represent the mean number of Tunel positive cells per section for the indicated structures (DRG, n=12; NT, n=6; and Vth Ganglion, n=5) +/−SEM.

(F-I) Survival of E10.5 dissociated trigeminal neurons cultured under neurotrophin-dependent conditions for 24 hr. Cell death was assessed by Tunel-staining (green), anti-NF-M staining (red), and nuclear stain (DAPI, blue).

(J) Quantitation of the number of Tunel-positive cells in the cultures shown (F-I). The mean=SEM of triplicate cultures, scored blindly, is shown.

(K-N) Wild-type E10.5 trigeminal explants were grown for 48 hr in a three-dimensional collagen matrix. Axonal outgrowth was visualized by anti-NF-M staining; (K), non-treated control; (L), cultures without NT-3 and NGF; (M), FK/CaA+NT throughout the culture period; (N), cultures in which FK/CaA is washed out after a period of 24 hr.

Scale bar for (A) through (D) is 50 μm; and for (M and L) 200 μm.

FIG. 5: illustrates that the inhibition of neurite outgrowth by calcineurin inhibition occurs after a several hour delay.

Small cubes of wild-type trigeminal explants were cultured on laminin, and growth cones were visualized by staining for F-actin using phalloidin (red).

(A) control culture; (B) addition of FK/CsA at the onset of culturing; (C) short-trm treatment (30 min) with FK/CsA; (D) Sema-3A-30 min. Addition of FK/CsA at the onset of culturing prevents axonal elongation; very few growth cones are seen, and those that are usually in the proximity of the cell bodies (arrow in B). Short-term treatment (30 min) with FK/CsA has no effect on growth cone morphology (C) and does not lead to growth cone collapse in contrast to the response to Sema-3A (D). Scale bar for (A-D) is 20 μm.

(E) Graph shows the mean change in neurite length (Δ) relative to the 24 hr time point +/−S/D of FK506/CsA-treated and nontreated explants as a function of time (t) after drug addition (means were generated from three independent experiments, n=9 for FK/CsA and n=6 for nontreated explants). Neurite length was calculated by subtraction of neurite length measured after 24 hours of growth (before drug addition, timepoint 0) from neurite length at the indicated timepoints after drug addition.

FIG. 6: illustrates that neurotrophins regulate NFATc translocation and transcriptional activation.

(A) Neurotrophin-induced cytoplasmic-to-nuclear translocation of NF-ATc4 in cortical neurons. Cells were transfected with EGFP-NFATc4 plasmid (2 μg) for all imaging experiments. Top row: Representative epifluorescence images showing NF-ATc4 distribution (green) before stimulation (“NS”, left), following 30 min of stimulation with 100 ng/ml BDNF in the absence of FK506/CsA (“BDNF”, middle) and in the presence of FK506/CsA (“BDNF+FK/CsA”, right). Note the nuclear translocation of NFATc in the middle condition, as evidenced by the loss of the empty halo of green fluorescence seen in the left and right panels. Middle row: Nuclear translocation of NFATc4 (green) upon stimulation with 100 ng/ml NGF in cortical neurons that were co-transfected with 2 μg wild-type TrkA. The addition of FK/CsA inhibited the nuclear translocation of NFATc4 induced by NGF stimulation (right). Bottom row: staining for the co-transfected, epitope-tagged, wild-type Trk-A construct (red). Scale bar for (A) is 10 μm.

(B) Activation of NFAT-dependent transcription by BDNF in cortical neurons, assessed using a transfected NFAT-luciferase reporter plasmid. FK506/CsA completely blocks the activation of the NFAT-reporter by BDNF.

(C) Activity of TrkA mutants on NFAT-dependent transcription in cortical neurons treated for 18 hours with NGF. NGF does not stimulate NFAT dependent transcription in cortical neurons (column 1). Co-transfection of wild-type TrkA allows activation of the NFAT-reporter by NGF in cortical neurons (column 2). Mutation of either the SHC interaction site (F499, column 3) of TrkA or the PLCγ interaction site (F794, column 4) of TrkA significantly reduces the ability of NGF to elicit NFAT-dependent transcription.

FIG. 7: shows that inhibition of calcineurin specifically blocks netrin-dependent but not netrin-independent growth from dorsal spinal cord explants.

(A and C) Calcineurin inhibition has no e ffect on netrin-independent axon outgrowth from dorsal spinal cord explants in either collagen or matrigel. E13 rat spinal cord explants were cultured for 43 hours in collagen (A) or matrigel (C) with increasing concentrations of FK506/CsA.

(B and D) FK/CsA treatment blocks netrin-dependent commissural axon outgrowth in a dose-dependent manner. E13 rat spinal cord explants were cultured for 19 hours in collagen (B) or matrigel (D) in presence of netrin-1 (100 ng/ml) and increasing concentrations of FK506/CsA. The total axon bundle length per explant was measured from at least 10 explants obtained from two independent experiments. Images of representative anti-NF-M stained explants are shown below the relevant bar for each condition.

(E) Netrin activates NFAT-dependent transcription in E15.5 cortical neurons in a calcineurin and DCC-dependent manner. E15.5 cortical neurons were transfected with 2 μg of NFAT-luciferase reporter+4 μg empty vector (left three lanes), 2 μg NFAT-luciferase co-transfected with 2 μg DCC+2 μg empty vector (middle three lanes) or 2 μg NFAT-luciferase+2 μg DCC+2 μg of dominant negative (Dn) DCC (right three lanes). Stimulation with 200 ng/ml recombinant netrin-1 activates NFAT-dependent transcription, this transcriptional induction is blocked by either FK/CsA treatment at the time of stimulation or co-transfection of Dn DCC. Cartoon depicts wild type DCC and Dn DCC, which lacks the cytoplasmic domain of wild type DCC.

FIG. 8: shows that Ca²⁺, calcineurin and NFATc transduce signals for neurite outgrowth but not survival. Model of signaling by netrins and neurotrophins. Calcineurin and NFATc are essential for netrin- and neurotrophin-dependent neurite outgrowth but appear to have little or no role in neurotrophin-induced survival or rapid growth cone attraction or repulsion.

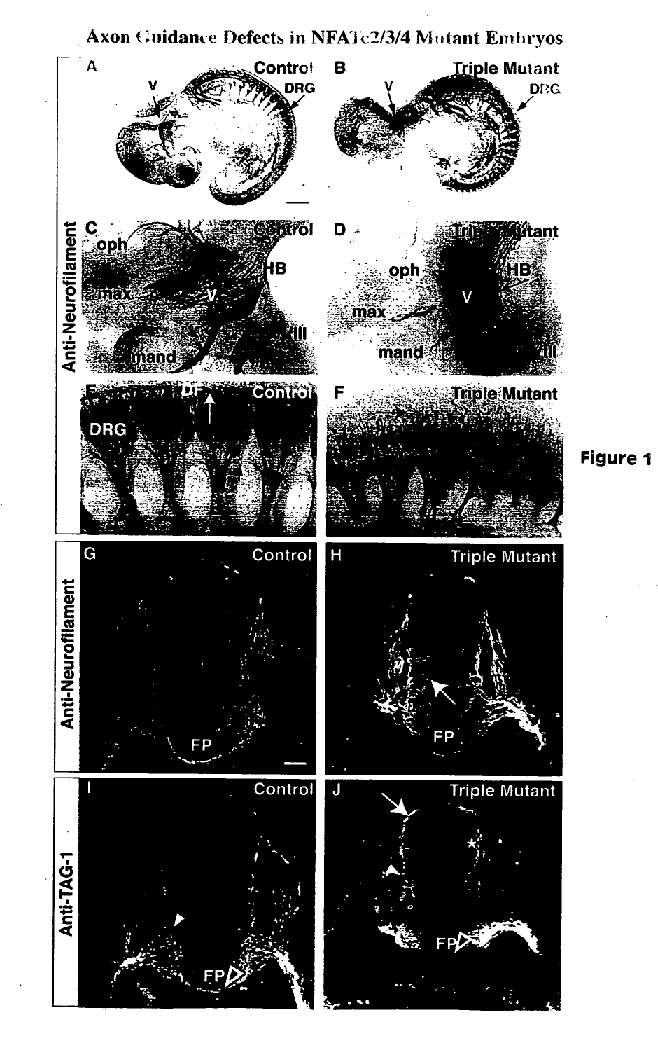

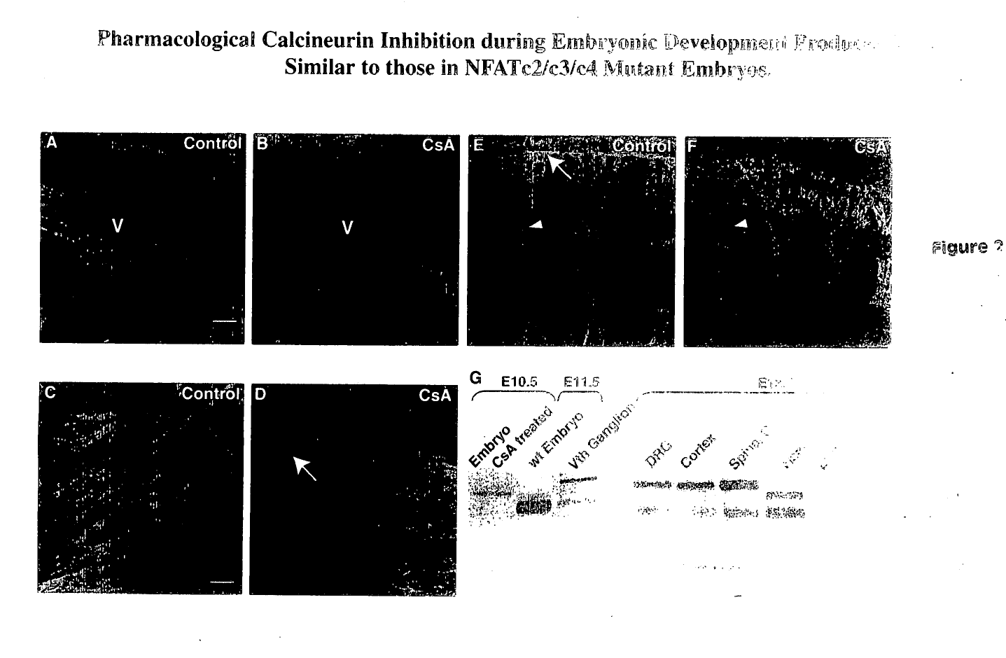

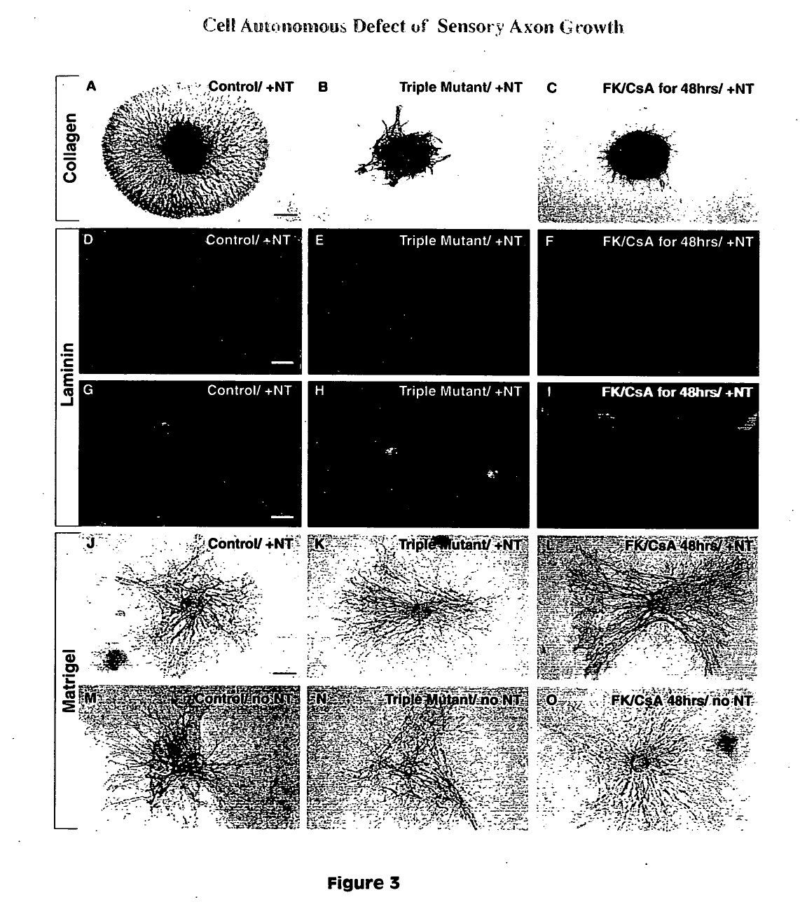

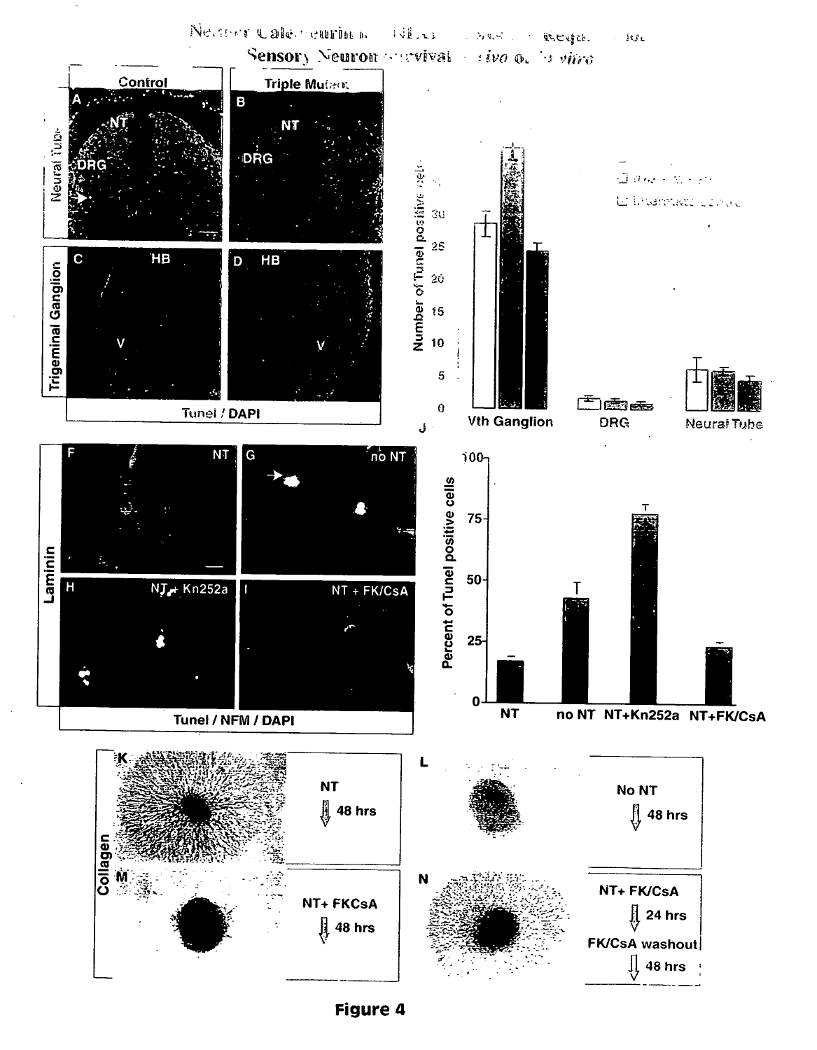

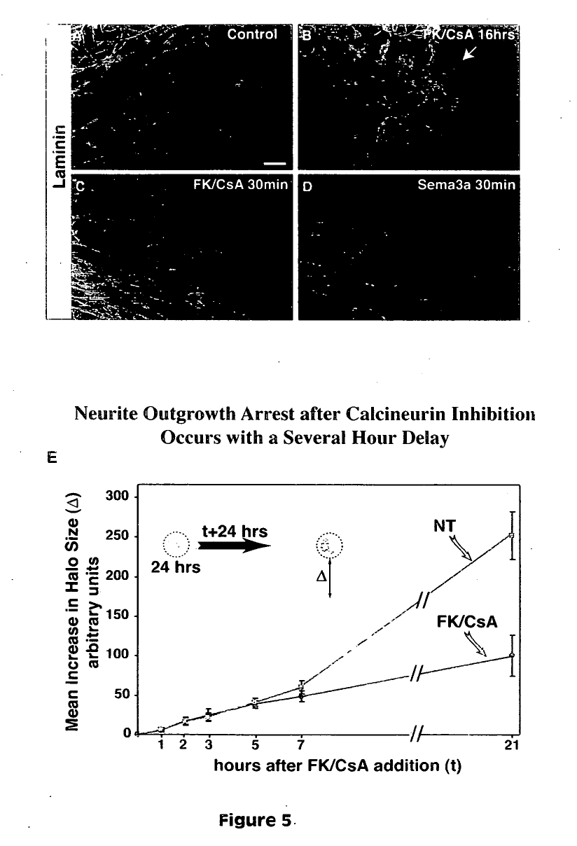

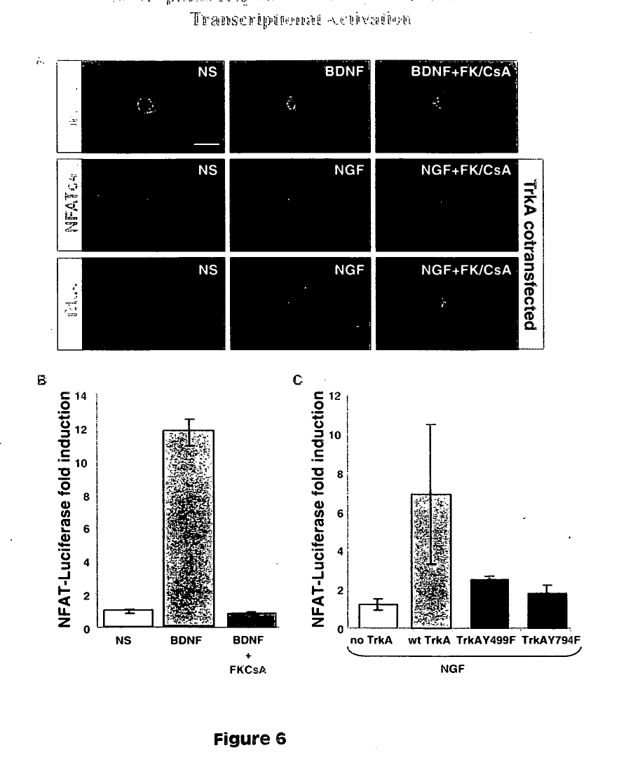

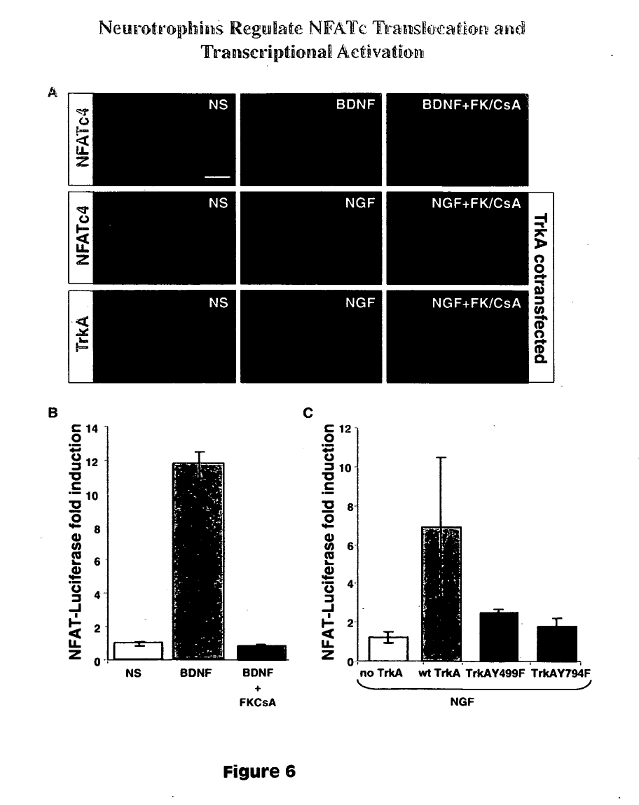

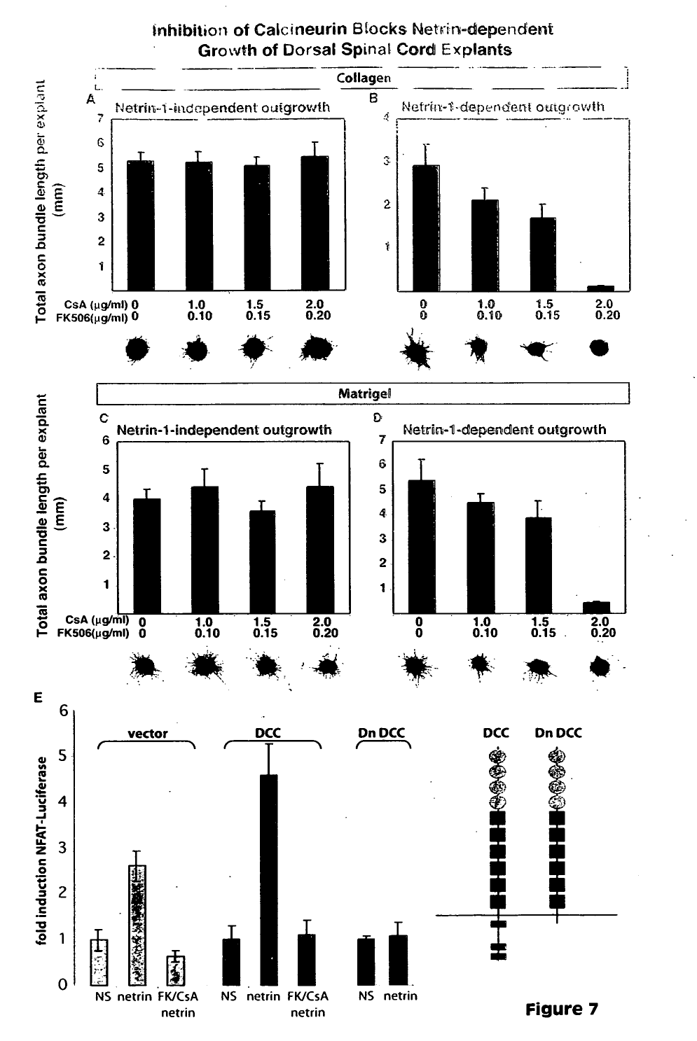

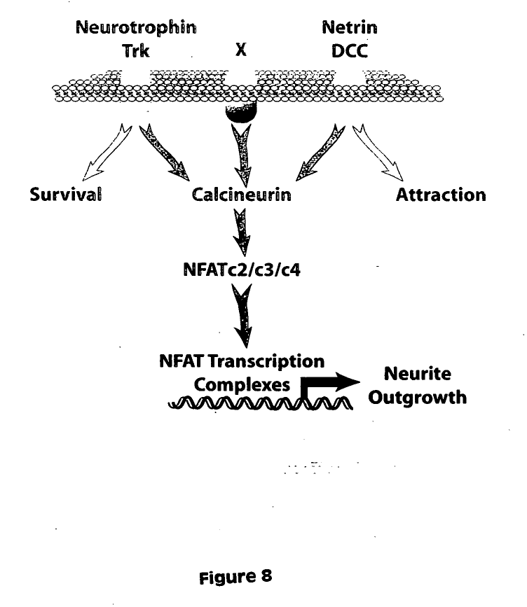

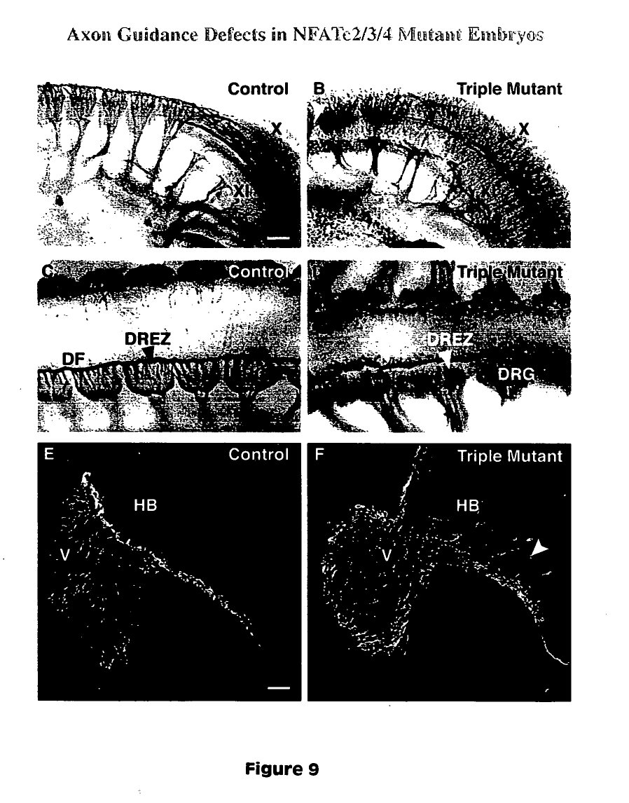

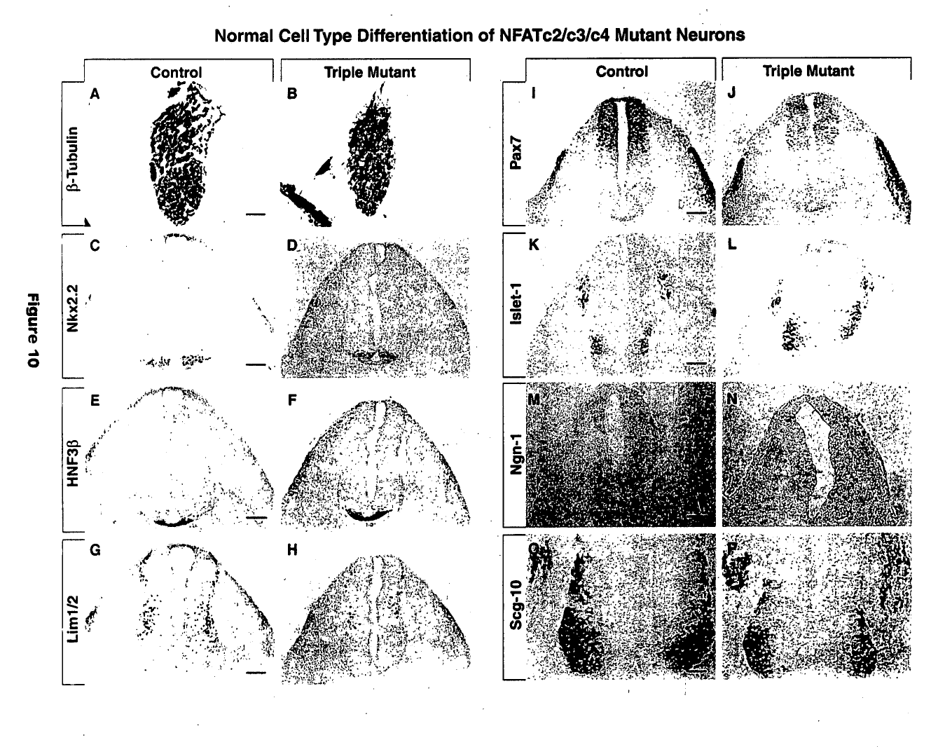

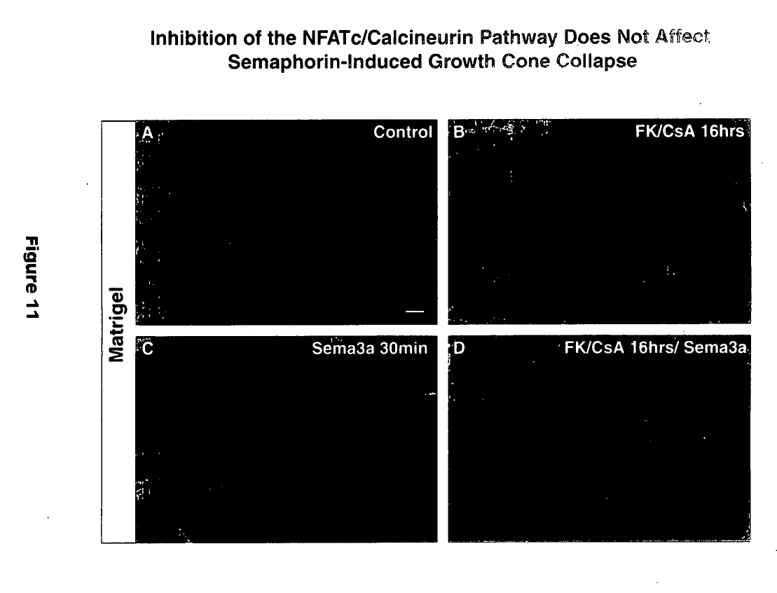

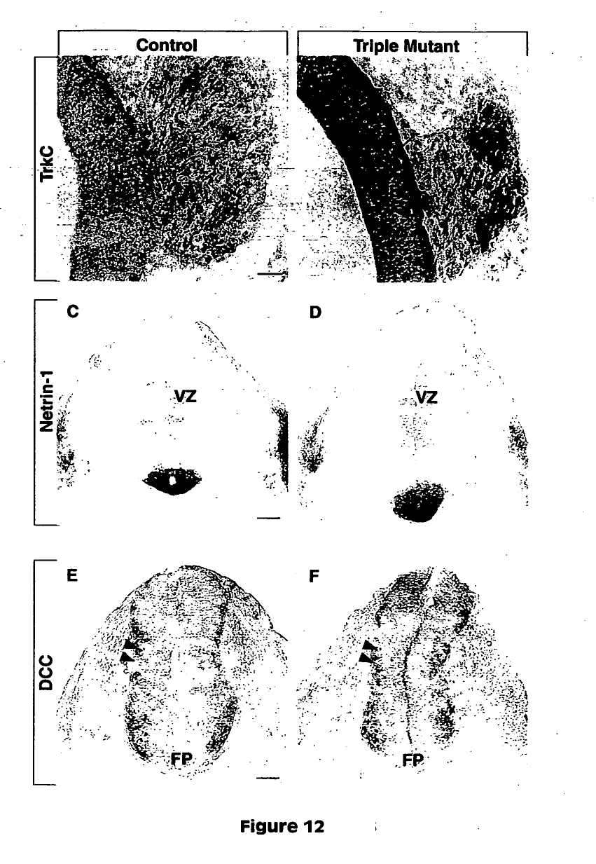

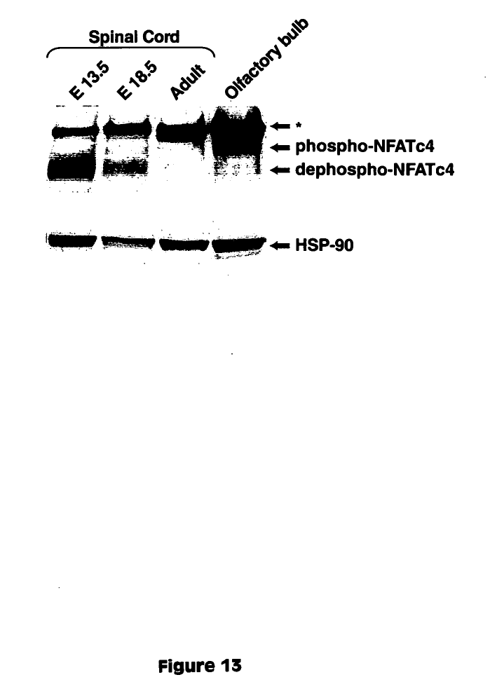

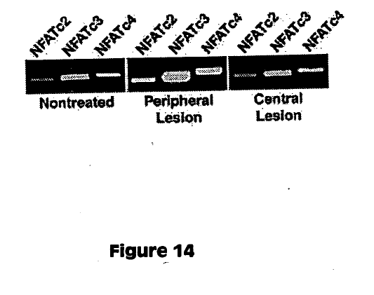

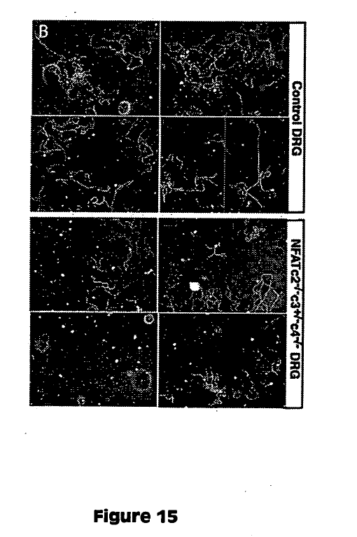

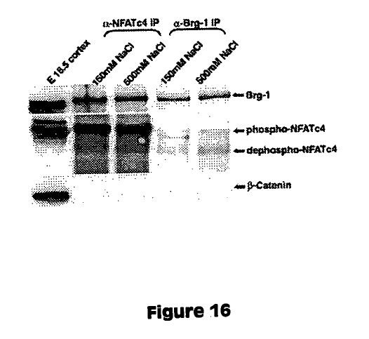

FIG. 9: Axon guidance defects in NFATc2/3/4 mutant embryos. (A-D) Whole-mount immunostaining with anti-neurofilament (NFM) antibody on E10.5 wild-type (A, C) and NFATc mutant (B, D) embryos. (A-B) At the hindbrain level, mutant embryos display defective axon trajectories with exuberant growth of central axons (B). The X^{th} cranial nerve (arrow) overshoots beyond the second cervical ganglion in the mutant embryo (white arrowhead in B), while it stops at the first cervical ganglion in the control embryo. (C-D) Dorsal view of the spinal cord. In littermate controls, sensory afferents from the DRG project to the dorsal root entry zone (DREZ, arrowhead in C) and send axons longitudinally in the dorsal funiculus (DF). In mutant embryos (D), these central processes from the DRG to the spinal cord fail to extend longitudinal tracts alongside the spinal cord and the DF is absent. However, an aberrant NFM positive structure (arrow in D), which is located medially of the DREZ (arrowhead in D), can be seen in the mutant embryos. This structure probably corresponds to the aberrant ventromedial projections of interneurons seen in FIG. 1H. (G-H) Transverse sections of E10.5 embryos at the level of the trigeminal ganglion stained with anti-NFM antibody show aberrant projections into the hindbrain of the mutant embryos (arrowhead in F). Dorsal is up. Scale bar for (A-B) is 800 μm, (C-D) is 500 μm and (E-F) is 50 μm.

FIG. 10: Immunocytochemical and in situ analysis of cell differentiation in NFATc mutant and control E10.5 embryos.

(A-B) Immunocytochemical detection of βIII-Tubulin in control (A) and mutant (B) trigeminal ganglion (V). Immunocytochemical staining of Nkx2.2 (C-D), HNF3β (E-F), Lim1/2 (G-H), Pax7 (I-J) and Islet-1 (K-L) in control and NFATc mutant spinal cord at E10.5. In situ analysis of neurogenin-1 (M-N) and Scg-10 (O-P) expression in control and NFATc mutant spinal cord at E10.5.

Dorsal is up. Scale bar for (A-B) is 50 μm, (C-N) 100 μm and for (O-P) is 50 μm.

FIG. 11: Inhibition of the NFATc/calcineurin pathway does not affect semaphorin induced growth cone collapse. Small cubes of E 10.5 wild-type trigeminal explants were cultured on matrigel, and growth cones were visualized by staining for F-actin using phalloidin (red). FK/CsA was added at the onset of culturing and left for the entire culturing period(B and D). Short-term treatment (30 min) with semaphorin 3-A induces growth cone collapse in both untreated (C) as well as FK/CsA treated (D) trigeminal neurons. Scale bar for (A-D) is 20 μm.

FIG. 12: (A-B) Immunocytochemical detection of Trk-C in control (A) and mutant (B) trigeminal ganglion (V) and hindbrain (HB). TrkC appears to be expressed at higher levels in the mutant embryo and aberrant TrkC positive projections (arrow in B) can be seen in the hindbrain. In situ analysis of netrin (C-D) expression in control and NFATc mutant spinal cord at E10.5. High level expression of netrin-1 is seen in the floor plate in both control and mutant embryos. Expression of netrin-1 in the ventricular zone (vz) (Serafini et al., 1996) in the mutant spinal cord is also comparable to the control. (E-F) DCC in situ hybridization analysis shows the expected pattern if expression in commissural neurons (arrowheads in E and F) in the spinal cord of mutant and control embryos. Dorsal is up. Scale bar for (A-B) is 50 μm, (C-F) 100 μm

FIG. 13: Shows a Western blot shows the downregulation of NFATc4 expression in adult neurons.

FIG. 14: Shows that induction of NFATc2, c3 and c4 appear in DRGs after transection of the sciatic nerve.

FIG. 15: L4/L5 DRGs from NFATc2^{-/-}, c3^{+/-},c4^{-/-} mutant mice show a reduction in axon outgrowth after sciatic nerve transaction.

FIG. 16: Shows that endogenous NFATc4 interacts tightly with endogenous Brg-1 protein in primary embryonic cortical neurons.

### DETAILED DESCRIPTION OF THE INVENTION

I. Overview

Neurite outgrowth is the first critical step of the process by which axons eventual reach their destinations. The present invention is based on the observation of extensive defects in axon outgrowth in NFATc3/c4 double mutant and NFATc2/c3/c4 triple mutant mice, as well as in mice treated during development with the highly specific calcineurin inhibitors, FK506 and CsA. Defects in peripheral extension of sensory axons may be explained by the finding that calcineurin/NFAT signaling is essential for neurotrophin-induced axon outgrowth. Surprisingly, NFAT signaling appears to have little role in neurotrophin-induced survival. Defects in commissural axons in NFATc null mice likely reflect a second requirement of calcineurin/NFAT signaling in mediating netrin-dependent outgrowth. NFAT target genes include cytoskeletal regulators such as the components of the Arp2/3 complex known to be essential for neurite outgrowth. The results described herein demonstrate a requirement for signaling by Ca²⁺, calcineurin and NFATc in a subset of developmental axon outgrowth programs required for wiring the embryonic nervous system, and indicate that agents that potentiate or otherwise activate NF-AT dependent gene transcription may be useful in the treatment of various disorders in which axon regeneration is desired.

NFAT transcription complexes are interesting candidates for regulating aspects of neuronal morphogenesis because they function as integrators of extracellular signals. Cell membrane signaling in a number of cell types results in the assembly of NFAT transcription complexes in the nucleus and the activation of sets of genes that are dependent on the cell type in which the signal is received (Crabtree and Olson, 2002; Shaw et al., 1988). A rise in intracellular Ca2+ to a threshold of about 400 nM (Klee et al., 1979), activates the serine/threonine phosphatase calcineurin and induces the rapid dephosphorylation of the cytoplasmic subunits, NFATc1-4 (HUGO Genome Nomenclature Committee, 1999) (Clipstone and Crabtree, 1992; Flanagan et al., 1991). Dephosphorylation of serines in the amino-termini of NFATc proteins by calcineurin exposes nuclear localization sequences leading to their rapid nuclear import (Beals et al., 1997a; Okamura et al., 2000; Zhu et al., 1998). NFAT cytoplasmic subunits require for DNA binding, other transcription factors including AP-1, MEF2, GATA4 and additional factors generically referred to as nuclear partners (NFATn) (Flanagan et al., 1991; Jain et al., 1993). The nuclear components of NFAT transcription complexes are often regulated by the PKC and Ras/MAPK pathways (Flanagan et al., 1991). Hence, the assembly of NF-AT transcription complexes requires that Ca2+/calcineurin signaling be coincident with other signaling pathways (Crabtree, 1989). Nuclear import of NFATc family members is opposed by rapid export of the proteins induced by rephosphorylation mediated by the sequential actions of PKA and GSK3 (Beals et al., 1997b). In addition, other kinases appear to oppose calcineurin-dependent import (Porter et al., 2000; Zhu et al., 1998). The rapid export of NFATc proteins from the nucleus makes NFAT signaling rapidly responsive to receptor occupancy and/or Ca2+channel dynamics (Dolmetsch et al., 1997; Graef et al., 1999; Timmerman et al., 1996).

The examples appended below provide evidence that NFATc family members function in the control of neuronal morphogenesis by conveying netrin and neurotrophin signals to the nucleus leading to the direct activation of genes essential for neurite outgrowth. Remarkably NFAT signaling appears to be specific for the neurite outgrowth, but not survival in response to neurotrophins, indicating that the major outcomes of neurotrophin signaling are due to the use of distinct biochemical pathways. The observation that this signaling pathway is also essential for patterning the embryonic vasculature is consistent with the growing notion that concerted mechanisms are used for the wiring the embryonic nervous system and patterning the vascular system.

II. Definitions

The terms “NF-AT,” “NFAT,” “NF-AT protein,” “NFATC,” and “NF-ATc” are used interchangeably herein. These terms refer to the family of nuclear factors of activated T cells. The GenBank Accession Numbers of exemplary human NF-AT nucleic acids and polypeptides are provided in the following Table:

| NF-AT | GenBank No. | |
|---|---|---|
| NF-ATc1 | | |
| NF-ATc | U08015 | |
| NF-ATc.b | U59736 | |
| NF-ATc2 | | |
| NF-AT1 | I38152 | |
| NF-ATp1 | U43341 | isoform B |
| | U43342 | isoform C |
| NF-ATc3 | | |
| NF-AT4a | I38155 | |
| NF-AT4b | I38156 | |
| NF-AT4c | L41067 | |
| NF-ATc4 | | |
| NF-AT3 | L41066 | |
| | I38154 | |
| NF-ATx | U14510 | |
| NF-ATx2 | U85428 | |
| NF-ATx3 | U85429 | |
| NF-ATx4 | U85430 | |

NF-ATc2 has also been referred to as NFIL2E and NFII-a.

Other examples of NF-AT genes and genes products can be found in GenBank, particularly accessions I80836, U36576, U36575, I60722, U02079, AF049606, AF087434, as well as PRF locus 2013343A, PIR locus S45262 and A48753. Exemplary NFAT polypeptides and nucleic acids are also disclosed in U.S. Pat. Nos. 6,388,052, 6,352,830, 6,312,899, 6,197,925, 6,171,781, 6,150,099, 6,096,515, and 5,837,840.

NF-AT is a transcription factor that remains cytosolic when phosphorylated. When cell stimulation results in an increase in intracellular calcium the heterodimeric serine/threonine phosphatase calcineurin is activated. Calcineurin dephosphorylates NF-AT, which then translocates to the nucleus and binds to specific regions in the promoters of some genes. This nuclear import and activation of NF-AT is opposed by rephosphorylation of NF-AT by NF-AT kinases and subsequent nuclear export.

The term “NF-AT agonist” as used herein refers to any molecule which activates or potentiates NF-AT dependent gene transcription. Such agonists can accomplish this effect in various ways. For instance, NF-AT agonists include molecules that can cause or promote a conformational change in an NF-AT protein such that NFAT remains localized in the nucleus. For instance, one class of agonists will increase the amount of NF-AT that is localized to the nucleus, such as by potentiating dephosphorylation of NF-AT, or promoting conformational changes resulting from dephosphorylation of NF-AT. Still another class of agonists can increase NF-AT transcriptional activity by activating phosphatases that act on NF-AT, such as calcineurin. Still other agonists inhibit phosphorylation of NF-AT by inhibiting kinases that act on NF-AT, such as GSK-3, PKA, or DRYK1A. Constitutively active (e.g., constitutively nuclear) NF-AT proteins or transcriptionally active fragments are also useful agonists. Other agonists are described herein and will be apparent to those skilled in the art.

NF-AT agonists include, but are not limited to, molecules that: (1) interact directly with NF-AT and modulate its nuclear translocation and activity; (2) interact directly with calcineurin and increase the dephosphorylation and/or activation of NF-AT; (3) interact directly with calmodulin and increases the activity of calcineurin and the dephosphorylation and/or activation of NF-AT; (4) stimulate an increase in intracellular calcium concentration which induces the activation of calcineurin and the dephosphorylation of NF-AT; (5) bind to a cell surface receptor and induce an increase in intracellular calcium concentration which induces the activation of calcineurin and the dephosphorylation of NF-AT; (6) interact with and inhibits GSK3 or other NF-AT kinases which functions to increase the nuclear duration and activity of NF-AT; (7) modify the DNA interaction of NF-AT in order to increase NF-AT dependent transcription; or (8) modify the interaction of NF-AT with a nuclear partner that results in an increase in transcription. An NF-AT agonist may also be a molecule which increases or enhances the expression of NF-AT.

By the term “effective amount” or “therapeutically effective amount” of an NF-AT agonist is meant an amount of an NF-AT agonist sufficient to obtain the desired physiological effect, e.g., regeneration of axons and/or decreased rate of loss of axons. An effective amount of an NF-AT agonist is determined by the care giver in each case on the basis of factors normally considered by one skilled in the art to determine appropriate dosages, including the age, sex, and weight of the subject to be treated, the condition being treated, and the severity of the medical condition being treated.

The term “GSK3” and “GSK-3” and are used interchangeably in this application.

III. Exemplary Embodiments

A. Exemplary Uses of NF-AT Agonists

Pharmaceutical compositions of NF-AT agonists may be used to promote nerve regeneration or to reduce or inhibit secondary nerve degeneration which may otherwise follow primary CNS or PNS injury, e.g., trauma (e.g., blunt trauma, penetrating trauma), compression [e.g., compression due to tendons and/or inflamed synovial membrane such as in carpal tunnel syndrome], bones [for instance sciatica], or growths [benign or cancerous, including growth of the nerves themselves or of surrounding tissue]) hemorrhagic stroke, ischemic stroke or damages caused by surgery such as tumor excision. In certain embodiments, NF-AT agonists may be used to treat spinal cord injuries.

Pharmaceutical compositions of NF-AT agonists may be used to treat any nervous system degenerative disorder. Nervous system degenerative disorders include, but are not limited to, Parkinson's disese, Alzheimer's disese, Huntington's Disease, Amyotrophic Lateral Sclerosis (ALS or Lou Gehrig's disease) and Multiple Sclerosis.

Pharmaceutical compositions of NF-AT agonists may be used to treat any peripheral neuropathy. Conditions associated with peripheral nerve damage include the following: alcoholism, amyloidosis, autoimmune disorders (e.g., Buillain-Barre syndrome), Bell's palsy, Carpal Tunnel Syndrome, chronic kidney failure, connective tissue disease (e.g., rheumatoid arthritis, lupus, sarcoidosis), diabetes mellitus, infectious disease (e.g., Lyme disease, HIV/AIDS, hepatitis B, meningitis, leprosy), liver failure, radiculopathy and vitamin deficiencies (e.g., pernicious anemia). Thus, NF-AT agonists may be used to treat peripheral neuropathies caused by any of the above mentioned conditions.

In certain embodiments, the pharmaceutical compositions of NF-AT agonists may be used as part of a therapeutic treatment program for motor neuropathies. Such motor neuropathies include, but are not limited to: adult motor neuron disease, including Amyotrophic Lateral Sclerosis (ALS or Lou Gehrig's disease); infantile and juvenile spinal muscular atrophies, and autoimmune motor neuropathy with multifocal conduction block.

In another embodiment, the motor neuropathy results from chronic disuse. Such disuse atrophy may stem from conditions including, but not limited to: paralysis due to stroke, spinal cord injury, brain trauma or other Central Nervous System injury; skeletal immobilization due to trauma (such as fracture, sprain or dislocation) or prolonged bed rest.

In yet another embodiment, the motor neuropathy results from metabolic stress or nutritional insufficiency, including, but not limited to, the cachexia of cancer and other chronic illnesses, fasting or rhabdomyolysis, endocrine disorders such as, but not limited to, disorders of the thyroid gland and diabetes.

The motor neuropathy can also be due to a muscular dystrophy syndrome, including but not limited to the Duchenne, Becker, myotonic, Fascioscapulohumeral, Emery-Dreifuss, oculopharyngeal, scapulohumeral, limb girdle, and congenital types, and the dystrophy known as Hereditary Distal Myopathy. In a further embodiment, the muscle atrophy is due to a congenital myopathy, including, but not limited to Benign Congenital Hypotonia, Central Core disease, Nemaline Myopathy, and Myotubular (centronuclear) myopathy.

In addition, pharmaceutical compositions of NF-AT agonists may be of use in the treatment of acquired (toxic or inflammatory) myopathies. Myopathies which occur as a consequence of an inflammatory disease of muscle, include, but are not limited to polymyositis and dermatomyositis. Toxic myopathies may be due to agents including, but not limited to amiodarone, chloroquine, clofibrate, colchicine, doxorubicin, ethanol, hydroxychloroquine, organophosphates, perihexiline, and vincristine.

In addition, such pharmaceutical compositions of NF-AT agonists may be used to ameliorate the effects of disease that result in a degenerative process, e.g., degeneration occurring in either gray or white matter (or both) as a result of various diseases or disorders, including, without limitation: diabetic neuropathy, senile dementias, Alzheimer's disease, Parkinson's Disease, facial nerve (Bell's) palsy, glaucoma, Huntington's chorea, non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeicneuropathy, Giant axonal neuropathy, Refsum's disease, Fabry's disease, lipoproteinemia, etc.

Moreover, compositions of NF-AT agonists may be used in conjunction with nerve grafts.

B. Exemplary NF-AT Agonists

In certain preferred embodiments, the NF-AT agonists that are used in the subject methods are ones that promote nuclear localization of transcriptionally active NF-AT proteins. In some embodiments, the method of the present invention utilize molecules that change the allosteric conformation of NF-AT, such that NF-AT will be localized in the nucleus of a cell.

In certain embodiments, the methods of the present invention utilize NF-AT agonists that enhance the dephosphorylation of NF-AT. Such agonists include phosphotases such as calcineurin, and molecules that increase the activity or the expression of calcineurin.

In other embodiments, the methods of the present invention utilize NF-AT agonists that inhibit the phosphorylation of NF-AT. Such agonists include inhibitors of kinases such as GSK-3, PKA and DRYK1A.

In one embodiment, the NF-AT agonist is a netrin or a neurotrophin.

In another embodiment, the NF-AT agonist is not a netrin or a neurotrophin.

In certain preferred embodiments, the NF-AT agonist activates NF-AT-dependent gene transcription.

The NF-AT agonists that are used in the subject methods may be small organic molecules or other biological molecules such as nucleic acids or proteins. The NF-AT agonists that are used in the subject methods may be applied to the target cells, e.g., formulated to be taken up by the target neurons. Further, the NF-AT agonists of the invention may be introduced into the target cells by techniques known in the art. Such techniques include, without limitation, the use of fusion or chimeric proteins including peptides such as the N-terminal sequence of HIV, a fragment of antennapedia, fragment C of tetanus toxin (Francis et al., Brain Res. 995(1):84-96 (2004). Other delivery vehicles are described in the art. See, e.g., Goodnough et al., “Development of a delivery vehicle for intracellular transport of botulinum neurotoxin antagonists,” FEBS Lett. 513(2-3):163-8 (2002); Mata et al., “Targeted gene delivery to the nervous system using herpes simplex virus vectors,” Physiol. Behav. 77(4-5):483-8 (2003).

In one embodiment, the NF-AT agonist is a constitutively active NF-AT protein. A constitutively active NFAT protein may be a naturally occurring protein or a mutant. Constitutive active NFAT protein are known in the art. See, e.g., Neal and Clipstone, J. Biol. Chem. 278(19):17246-54 (2003); Porter and Clipstone, J. Immunol. 168(10):4936-45 (2002); Monticelli and Rao, Eur. J. Immunol. 32(10):2971-8 (2002); Plyte et al., J. Biol. Chem. 276(17):14350-58 (2001). Nucleic acids encoding constitutively active forms of NFAT can be introduced into the target cell by techniques known in the art, such as gene therapy. Further, constitutively active forms of NFAT can be applied to target cells using delivery techniques such as liposomes, or by forming chimeric proteins of a constitutively active NFAT protein that includes a fusogenic peptide such as the N-terminal sequence of HIV-TAT protein or a fragment of antennapedia.

In certain embodiments, the methods of the present invention utilize NF-AT agonists that enhance the activity of calcineurin. For instance, the activity of calcineurin can be enhanced or increased through introduction of a gene that expresses calcineurin or a protein that upregulates the expression of calcineurin or a protein that prevents the downregulation of calcineurin (such as MCIPs). The introduction of a gene (an endogenous gene that has been altered, or a gene originally isolated from a different organism, for example) into cells, either in vitro or in a patient, can be accomplished by any of several known techniques, for example, by vector mediated gene transfer, as by amphotropic retroviruses, calcium phosphate, or liposome fusion, for example.

A gene intended to have an effect on neurons in a host mammal can be delivered to isolated neuronal cells by the use of viral vectors comprising one or more nucleic acid sequences encoding the gene of interest. Generally, the nucleic acid sequence has been incorporated into the genome of the viral vector. In vitro, the viral vector containing the nucleic acid sequences encoding the gene can be contacted with a cell and infection can occur. The cell can then be used experimentally to study, for example, the effect of the gene on growth of neuronal cells in vitro or the cells can be implanted into a patient for therapeutic use. The cells to be altered by introduction or substitution of a gene can be present in a biological sample obtained from the patient and used in the treatment of disease, or can be obtained from cell culture and used to dissect developmental pathways of arteries and veins in in vivo and in vitro systems.

After contact with the viral vector comprising a nucleic acid sequence encoding the gene of interest, the treated neuronal cells can be returned or readministered to a patient according to methods known to those practiced in the art. Such a treatment procedure is sometimes referred to as ex vivo treatment. Ex vivo gene therapy has been described, for example, in Kasid, et al., Proc. Natl. Acad. Sci. USA 87:473 (1990); Rosenberg, et al., New Engl. J. Med. 323:570 (1990); Williams, et al., Nature 310476 (1984); Dick, et al., Cell 42:71 (1985); Keller, et al., Nature 318:149 (1985); and Anderson, et al., U.S. Pat. No. 5,399,346 (1994).

Generally, viral vectors which can be used therapeutically and experimentally are known in the art. Examples include the vectors described by Srivastava, A., U.S. Pat. No. 5,252,479 (1993); Anderson, W. F., et al., U.S. Pat. No. 5,399,346 (1994); Ausubel et al., “Current Protocols in Molecular Biology”, John Wiley & Sons, Inc. (1998). Suitable viral vectors for the delivery of nucleic acids to cells include, for example, replication defective retrovirus, adenovirus, parvovirus (e.g., adeno-associated viruses), and coronavirus. Examples of retroviruses include avian leukosis-sarcoma, mammalian C-type, B-type viruses, lentiviruses (Coffin, J. M., “Retroviridae: The Viruses and Their Replication”, In: Fundamental Virology, Third Edition, B. N. Fields, et al., eds., Lippincott-Raven Publishers, Philadelphia, Pa., (1996)). The mechanism of infectivity depends upon the viral vector and target cell. For example, adenoviral infectivity of HeLa cells occurs by binding to a viral surface receptor, followed by receptor-mediated endocytosis and extrachromasomal replication (Horwitz, M. S., “Adenoviruses” In: Fundamental Virology, Third Edition, B. N. Fields, et al., eds., Lippincott-Raven Publishers, Philadelphia, Pa., (1996)).

Instead of gene therapy, a calcineurin protein or a molecules that activates a calcineurin protein can be applied to the target cells, e.g., formulated to be taken up by the target neurons.

In one embodiment, the methods of the present invention utilize NF-AT agonists that inhibit a modulatory calcineurin-interacting protein (MCIP). In one embodiment, the NF-AT agonist to be used in the claimed methods is the pyridine activator of myocite hypertrophy (“PAMH”) disclosed in Bush et al., PNAS, 101(9):2870-2875.

Another embodiment of the invention relates to methods for decreasing the level of NF-AT phosphorylation in a mammal by administering an agent which down-regulates gene expression of GSK3 or other kinases that phosphorylate NF-AT proteins. GSK3 inhibitors include agents that inhibits expression of GSK3 (such as antisense or RNAi constructs), agents that act upstream of GSK3 and downregulate its expression, stability and or activation as a kinase, as well as pharmacological inhibitors of GSK3, such as small organic molecules that bind to and inhibit the kinase activity of GSK3. A preferred agent is a nucleic acid, such as an antisense nucleic acid or an RNA interference (RNAi) construct. Optionally, the agent is a small molecular compound. In certain cases, axonal growth may be promoted when the agent reduces gene expression of GSK3. International Patent Applications Publication Numbers WO 02/062387, WO 00/21927, WO 00/386755 WO 01/09106 and WO 01/74771 (SmithKline Beecham PLC), WO98/16528 and U.S. Application Nos. 2004/0024040 and 2004/0019052 disclose certain compounds useful as GSK-3 inhibitors. The teachings of those publications are incorporated by reference herein

For example, the invention contemplates the use of antisense nucleic acid corresponding to a portion of a gene encoding a GSK3 polypeptide, which antisense decreases the level of expression of the GSK3 protein. Such an antisense nucleic acid can be delivered, for example, as an expression plasmid which, when transcribed in the cell, produces RNA which is complementary to at least a unique portion of the cellular mRNA which encodes GSK3. Alternatively, the construct is an oligonucleotide which is generated ex vivo and which, when introduced into the cell causes inhibition of expression by hybridizing with the mRNA and/or genomic sequences encoding GSK3. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

In certain aspects, the invention relates to the use of RNA interference (RNAi) to effect knockdown of GSK3 or other kinases which phosphorylate NF-AT. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. RNAi constructs can comprise either long stretches of dsRNA identical or substantially identical to the target nucleic acid sequence or short stretches of dsRNA identical to substantially identical to only a region of the target nucleic acid sequence.

Optionally, the RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the method has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially a bolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

In some cases, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

The RNAi construct can also be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response.

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In other aspects, the method relates to the use of ribozyme molecules designed to catalytically cleave an mRNA transcripts to prevent translation of mRNA, such as GSK3 mRNA (see, e.g., PCT International Publication WO90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. The ribozymes of the present invention also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In a further aspect, the invention relates to the use of DNA enzymes to inhibit expression of GSK3. DNA enzymes incorporate some of the mechanistic features of both antisense and ribozyme technologies. DNA enzymes are designed so that they recognize a particular target nucleic acid sequence, much like an antisense oligonucleotide; however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

In addition to affecting the levels or activity of calcineurin and GSK3 using biological macromolecules, small organic molecules can also be used to increase the activity of calcineurin or decrease the activity of GSK3 in a patient, either in the affected tissues specifically or throughout a patient's tissues.

Compounds that activate, agonize, or mimic the activity of calcineurin are NF-AT agonists. These compounds include, but are not limited to, calcium ionophores, such as A23187 and ionomycin, angiotensin II, phenylephrine, 1% fetal bovine serum, carbachol, cholecystokinin (including the 26-33 fragment), and cholinergic agonists such as carbamylcholine.

Compounds that antagonize, inhibit, or suppress the activity of GSK3 are NF-AT agonists. These compounds include, but are not limited to, insulin, wnt proteins, MAPKAP-K1 (RSK), protein kinase B (Akt), paullones such as alsterpaullone (Leost et al., Eur. J. Biochem. 267:5983-94 (2000)), growth factor (GF), epidermal growth factor (EGF), lithium chloride, maleimides such as Ro 31-8220, SB 216763, and SB 415286, aloisines such as aloisines A and B, p70 ribosomal S6 kinase 1 (S6K1), cyclic AMP analogs and agonists, hymenialdisines such as dibromohymenialdisine, indirubins such as 5,5′dibromo-indirubin, muscarinic antagonists such as AF 150 and AF102B, and Frequently rearranged in advanced T-cell lymphomas 1 (FRAT1) (including the 188-226 fragment). GSK3 has recently been reviewed, S. Frame and P. Cohen, Biochem. J. (2001) 359, 1-16, and additional information about GSK3 has been surveyed, B. W. Doble and J. R. Woodget, J. Cell Sci. (2003) 116, 1175-1186.

In one embodiment the claimed methods use an NFAT agonist that is an agonist of peroxisome proliferator-activated receptor-gamma (“PPARgamma”). PPAR gamma agonists are well known to those of skill in the art and include, for example, thiozolidinediones (TZD). Particularly preferred PPARgamma agonists include, but are not limited to rosiglitazone, troglitazone (Resulin), farglitazar, phenylacetic acid, GW590735, GW677954, Avandia, Avandamet (avandia+metformin), ciglitazone, 15 deoxy prostaglandin J2 (15PGJ2), 15-deoxy-delta12,14 PGJ2, GW-9662, MCC-555 (disclosed in U.S. Pat. No. 5,594,016), analogues thereof and the like. PPAR gamma agonists include thiazolidinedione derivatives such as pioglitazone [(±)[[4-[2-(5-ethyl pyridinyl)ethoxy]phenyl]methyl]-2,4thiazolidinedione], troglitazone [(±)[[4-[(3,4-dihydro hydroxy-2,5,7,8-tetramethyl-2H benzopyran yl)methoxy]phenyl]methyl]-2,4-thiazolidinedione], ciglitazone [5-[[4-[(lmethylcyclohexyl)methoxy]phenyl]methyl]-2,4-thiazolidinedione, rosiglitazone [(±) [4-[2-[N-methyl-N-(2-pyridyl)amino]ethoxy]benzyl]-2,4-thiazolidinedione] and other 2,4thiazolidinedione derivatives as well as pharmaceutically suitable acid addition salts thereof. Other PPARgamma agonist include: S)-2-ethoxy-3-[4-(2-{4-methanesulphonyloxyphenyl}ethoxy-)phenyl]propanoic acid, WY-14643, clofibrate, fenofibrate, bezafibrate, GW 9578, englitazone (CP-68722, Pfizer), proglitazone, BRL-49634, KRP-297, JTT-501, SB 213068, GW 1929, GW 7845, GW 0207, L-796449, L-165041, GW 2433, GL-262570 (Glaxo Welcomes), darglitazone (CP-86325, Pfizer, isaglitazone (MIT/J&J), JTT-501 (JPNT/P&U), L-895645 (Merck), R-119702 (Sankyo/WL), NN-2344 or balaglitazone (Dr. Reddy/NN), or YM-440 (Yamanouchi). Other PPARgamma agonists include AZ-242/tesaglitazar (Astra/Zeneca; as described: in B. Ljung et. al., J. Lipid Res., 2002, 43, 1855-1863), GW409544 (Glaxo-Wellcome), KRP-297/MK-767 (Kyorin/Merck; as described in: K. Yajima et. al., Am. J. Physiol. Endocrinol. Metab., 2003, 284: E966-E971) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998) or the compounds (from Bristol-Myers Squibb) described in U.S. Pat. No. 6,414,002. Other PPARgamma agonist include GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchrom-ane-2-carboxylic acid (disclosed in U.S. Ser. Nos. 60/235,708 and 60/244,697).

In other embodiments, the methods use an NFAT agonist that upregulates AP-1 in motormeurons. In one embodiment, AP-1 is upregulated by a PACAP, a Maxadilan, a PACAP receptor agonist, or a ADCYAP1R1 agonist.

C. Screening Assays to Identify NF-AT Agonists

The invention also provides screening assays for identifying compounds which inhibit phosphorylation of a NF-AT protein or increase depohosphorylation of an NF-AT protein. In this regard, an NF-AT agonist is an agent which either inhibits phosphorylation of an NF-AT protein, or potentiates dephosphorylation of an NF-AT protein. In certain embodiments of the assay, it may be desirable to directly detect changes in phosphorylation of an NF-AT protein.

In one embodiment, the assay is an in vitro assay. In one embodiment, the assay comprises contacting a non-phosphorylated, or partially phosphorylated NF-AT protein with a cell extract, or with one or more purified kinases, such as GSK-3, PKA and DRYK1A, and other necessary components of an in vitro kinase assay, including a source of phosphate and with or without a test compound and under conditions under which phosphorylation of NF-AT occurs. The comparison of the state of phosphorylation of NF-AT in the presence and in the absence of a test compound will indicate whether the test compound decreases or inhibits the phosphorylation of NF-AT.

In another embodiment, the kinase assay is an in vivo kinase assay. The assay can comprise incubating a cell expressing non-phophorylated or partially phosphorylated NF-AT, e.g, an activated T cell, with a test compound and comparing the state of phosphorylation of NF-T in the presence and in the absence of the test compound. A variation in the state of phosphorylation will indicate that the test compound is capable of modulation phosphorylation of NF-AT. The state of phosphorylation of NF-AT can be determined by, e.g., by performing the incubation of the cells in the presence of labeled, e.g., radioactive, phosphate (e.g., ATP), and determining the amount of label present in an immunoprecipitate with an NF-AT specific antibody. Alternatively, the state of phosphorylation can be performed by Western blot analysis, optionally coupled with immunoprecipitations.

In another embodiment, the invention provides screening assays for identifying compounds which increase dephosphorylation of NF-AT, such as activators of calcineurin-mediated dephosphorylation of an NF-AT protein. In one embodiment, the assay comprises incubating a phosphorylated NF-AT protein with a cell extract or with one or more phosphatases, e.g., calcineurin, in conditions under which the NF-AT polypeptide can be dephosphorylated, and a test compound. The NF-AT protein can be phosphorylated in vitro with PKA and optionally GSK-3, or it can be phosphorylated with a cell extract. NF-AT can also be isolated from or present in a cell extract. The comparison of the state of phosphorylation of NF-AT after a phosphatase reaction in the presence and in the absence of a test compound will indicate whether the test compound is capable of increasing dephosphorylation of NF-AT, and therefore be a candidate NF-AT agonist. The state of phosphorylation of NF-AT can be determined as described above.

In yet another embodiment, the drug screening assay is derived to include a whole cell expressing an NF-AT protein. For instance, the level of an intracellular second messenger responsive to activities dependent on an NF-AT protein can be detected. For example, in various embodiments the assay may assess the ability of test agent to cause changes in or expression of genes whose transcription is dependent on an NF-AT protein. By detecting changes in intracellular signals, such as alterations in second messengers or gene expression, candidate agonists of NF-AT -dependent signaling can be identified.

By selecting transcriptional regulatory sequences from target genes, e.g., NF-AT dependent transcriptional control elements, and operatively linking such promoters to a reporter gene, the present invention provides a transcription based assay which is sensitive to the ability of a specific test compound to influence signaling pathways dependent on an NF-AT protein.

In an exemplary embodiment, the subject assay comprises detecting, in a cell-based assay, change(s) in the level of expression of a reporter gene controlled by a transcriptional regulatory sequence responsive to signaling by an NF-AT protein. Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on signaling by the NF-AT protein. Expression of the reporter gene, thus, provides a valuable screening tool for the development of compounds that act as agonists or antagonists of NF-AT protein-dependent signalling. The reporter gene may be a luciferase gene. See Example 6. The use of transcription based assays is well known in the art.

The invention further provides screening assays for identifying compounds which increase nuclear localization of an NF-AT protein. The screening assays can be in vivo or in vitro and can be cell based or based on a cell free format. In a preferred embodiment, the assays allow the identification of compounds which increase NF-AT translocation across the nuclear membrane. In certain embodiments, the translocation of NF-AT across the nuclear membrane is detected using immunofluorescence. See Example 6.

D. Combination Therapy

In one embodiment, the methods described in this application involve the use of an NF-AT agonist in combination with another agent or component.

In one embodiment, the additional agent or component is another NF-AT agonist.

In one embodiment, the additional agent or component can be a compound that promotes nerve growth or nerve regeneration or axonal regeneration or axonal growth. In one embodiment, the additional agent can be a neurotrophic factor. Neurotrophic factors include, but are not limited to nerve growth factor (NGF), BDNF, and glial cell line-derived neurotrophic factor (GDNF), neurturin, neurotrophin-, neurotrophin-4, neurotrophin-5, neurotrophin-6 and other related neurotrophins. In yet another embodiment, the other agent or component can be a netrin.

In one embodiment, the additional agent and component can be inosine.

In yet another embodiment the additional agent and component can be a neuropoietic factor which has an effect on both brain and in myeloid cells (e.g., cholinergic differentiation factor [also called leukemia inhibitory factor], ciliary neurotrophic factor (CNTF), oncastatin M, growth promoting factor, sweat gland factor, and the interleukins 6 and 11).

In another embodiment, the additional agent and component can be a fibroblast growth factor, an insulin-like growth factor or a platelet-derive growth factor.

In another embodiment, the additional agent and component can be an anti-inflammatory. In one embodiment, the anti-inflammatory is a nonsteroidal antiinflammatory drug (NSAID) that inhibits the enzyme, cyclooxygenase (COX). In one embodiment, the NSAIDs include selective COX-2 inhibitors such as celocoxib (Celebrex®), refocoxib (Vioxx®), and N-[2-(cyclohexyloxy)-4-nitrophenyl]methanesulfonamide (NS-398).

In another embodiment, the additional agent and component can be anti-NGF, anti-BDNF and/or anti-IGF-I and/or other antibodies that can be used to reduce unwanted “sprouting”, to reduce post-transectional collateral axonal branching.

In another embodiment, the additional agent and component can be transforming growth factor-beta 1 (TGF-beta 1), or other agents that increase production of inducible-nitric oxide synthase (i-NOS).

In another embodiment, the a dditional agent and component can be an activator of a macrophage (such as lipopolysaccharide (LPS), or a combination of LPS and indomethacin). The combination of NFAT agonist and an activator of macrophage can reduce the degree of cavitation and increase the number of cells and axons in the lesion.

In another embodiment, the additional agent and component can be a leukemia inhibitory factor (LIF).

E. Pharmaceutical Compositions

The invention also comprises a pharmaceutical composition comprising a therapeutically effective amount of an NF-AT agonist and a pharmaceutically acceptable carrier.

In one embodiment, the NF-AT agonist is calcineurin or an activator of calcineurin. In another embodiment, the NF-AT agonist is an inhibitor of GSK3. In another embodiment, the NF-AT agonist is a neurotrophin. In another embodiment, the NF-AT agonist is a netrin. In another embodiment, the NF-AT agonist is a constitutively active NF-AT protein.

In certain embodiments, the pharmaceutical composition comprises an NF-AT and other components. The additional component may be another NF-AT agonist or another compound.

In one embodiment, the pharmaceutical composition of the invention comprises an NF-AT agonist and another compound that promotes nerve growth or nerve regeneration. In another embodiment, the pharmaceutical composition of the invention comprises an NF-AT agonists and another compound that promotes axonal regeneration or axonal growth. Factors known to enhance nerve regeneration include, but are not limited to, nerve growth factor (NGF), neuronotrophic factor (NTF), ciliary neuronotrophic factor NTF (CNTF), motor nerve growth factor (MNGF), fibronectin, neurite promoting factor (NPF), laminin, neural cell adhesion molecule (N-CAM), n-cadhenin, fibrin, matrix factor (MF), estrogen, testosterone, thyroid hormone, corticotropin, insulin, catalase, acidic fibroblast growth factor (aFGF), basic fibroblast growth factor (bFGF), forskolin, glia-derived protease inhibitor (GdNPF), GM-1 Gangliosides, isaxonine, leupeptin, and pyronin.

In one embodiment, the pharmaceutical composition of the invention comprises an NF-AT agonist as described above and a neurotrophic factor. Neurotrophic factors include, but are not limited to nerve growth factor (NGF), BDNF, and glial cell line-derived neurotrophic factor (GDNF), neurturin, neurotrophin-, neurotrophin-4, neurotrophin-5, neurotrophin-6 and other related neurotrophins. In yet another embodiment, the pharmaceutical composition of the invention comprises an NF-AT agonist and a netrin.

In one embodiment, the pharmaceutical composition of the invention comprises an NF-AT agonist and inosine.

In yet another embodiment, the pharmaceutical composition of the invention comprises an NF-AT agonist and a neuropoietic factor which has an effect on both brain and in myeloid cells (e.g., cholinergic differentiation factor [also called leukemia inhibitory factor], ciliary neurotrophic factor, oncastatin M, growth promoting factor, sweat gland factor, and the interleukins 6 and 11).

In another embodiment, the pharmaceutical composition of the invention comprises an NF-AT agonist and a fibroblast growth faction, an insulin-like growth factor or a platelet-derive growth factor.

In another embodiment, the pharmaceutical composition of the invention comprises an NF-AT agonist and an anti-inflammatory. In one embodiment, the anti-inflammatory is a nonsteroidal antiinflammatory drug (NSAID) that inhibits the enzyme, cyclooxygenase (COX). In one embodiment, the NSAIDs include selective COX-2 inhibitors such as celocoxib (Celebrex®), refocoxib (Vioxx®), and N-[2-(cyclohexyloxy)-4-nitrophenyl]methanesulfonamide (NS-398).

In another embodiment, the pharmaceutical composition of the invention comprises an NF-AT agonist and neutralizing concentrations of anti-NGF, anti-BDNF and/or anti-IGF-I and/or other antibodies that can be used to reduce unwanted “sprouting”, to reduce post-transectional collateral axonal branching.

In another embodiment, the pharmaceutical composition of the invention comprises an NF-AT agonist and transforming growth factor-beta 1 (TGF-beta 1), or other agents that increase production of inducible-nitric oxide synthase (i-NOS).

In another embodiment, the pharmaceutical composition of the invention comprises an NF-AT agonist and an activator of a macrophage (such as lipopolysaccharide (LPS), or a combination of LPS and indomethacin). The combination of NFAT agonist and an activator of macrophage can reduce the degree of cavitation and increased the number of cells and axons in the lesion.

In another embodiment, the pharmaceutical composition of the invention comprises an NF-AT agonist and leukemia inhibitory factor (LIF).

The pharmaceutical compositions of the invention may be formulated for administration in any convenient way for use in human or veterinary medicine. The pharmaceutical compositions of the invention include those suitable for oral/nasal, topical, and/or parenteral administration. The pharmaceutical compositions of the invention may conveniently be presented in unit dosage form and may be prepared by any methods well known in the art of pharmacy.

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents.

Administration can be systemic or local.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, care must be taken to use materials to which the protein does not absorb.

In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug B ioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J.Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In one embodiment, the pharmaceutical composition is administered locally to the desired location. For example, in one embodiment the composition is administered into the subarachnoid space after spinal cord injury. In another embodiment, the composition is introduced into the cerebrospinal fluid of the subject. In certain another embodiment, the composition is introduced intrathecally, e.g., into a cerebral ventricle, the lumbar area, or the cistema magna. In another embodiment the composition is introduced intraocullarly, to thereby contact retinal ganglion cells.

In another embodiment the composition is delivered locally to promote guided neurite elongation. Such methods are well known in the art, and include the use of synthetic nerve conduits, preferably permeable biodegradable tubes such as those prepared from collagen. See, e.g., Meaha et al., J. Neurosurg. 78, 90 (1993); Doolabn et al., Rev. Neurosci. 7(1):47-84 (1996); Lee and Wolfe, J. Am. Acad. Orthop. Surg. 8(4):243-52 (2000).

In another embodiment, the composition can be used as part of an entubulation treatment, e.g., using collagen or other biodegradable tubes as conduits, and including NF-AT agonists (alone or in combination with other agents) in a permeable matrix provided within the tube. Exemplary formulations include NF-AT agonist provided with fibronectin and laminin to promote axon growth in the entubulation model.

In another embodiment, the composition can be use implanted in a prosthesis. For example the composition can be implanted in neural prosthetic devices used in entubulation methods of repairing (regenerating) nerves. Such methods are well known in the art and have been described in publications including, but not limited to, U.S. Patent Application Nos. 20030028204 and 20020018799.

In another embodiment, the compositions are preferably administered to target the tissues of the CNS by direct infusion into the CNS or cerebrospinial fluid, conjugation with a molecule which naturally passes into the CNS, by reducing the overall length of the polypeptide chain and retaining the biologically active site, or by increasing the lipophilicity of the compounds, e.g., by appropriate amino acid substitutions.

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The amount of active ingredient(s) which can be combined with a carrier material to produce a single dosage form will vary depending upon the host being treated, the particular mode of administration. The amount of active ingredient(s) which can be combined with a carrier material to produce a single dosage form will generally be that amount of the compound(s) which produces a therapeutic effect.

### EXAMPLES

### Example 1

Mice Bearing Mutations in NFATc2, c3 and c4 have Defects in Axon Outgrowth

Profound defects in sensory axon projections were observed in embryos with combined deletions of either NFATc3 and NFATc4 (c3/c4 mutants) or of NFATc2, NFATc3 and NFATc4 (c2/c3/c4 mutants) using neurofilament (NFM) staining at E10.5 (FIG. 1A-D and FIG. 10). Defects were seen in about 70% of c3/c4 mutants and 100% of c2/c3/c4 mutants, but the nature of the defects was similar. No defects in axonal projections were observed at this level of analysis in the single mutants (data not shown). In what follows, we focus on analysis of the triple c2/c3/c4 mutants. The triple mutant embryos are smaller than stage-matched control littermates but were at the same Theiler stage, and were not developmentally delayed (FIG. 1). The smaller size is likely due to the requirement for calcineurin/NFAT signaling in patterning the vertebrate vasculature (Graef et al., 2001a). Vascular defects often accompany mutations in axonal guidance molecules, apparently reflecting common requirements for patterning the nervous and vascular systems (Behar et al., 1996; von Schack et al., 2001).

At E10.5 (embryonic day 10.5), most peripheral trigeminal axons observed in the c2/c3/c4 embryos were stunted, but neurite outgrowth appeared to initiate in the correct direction for cranial and dorsal root ganglia (FIGS. 1A-1F). NFM staining was consistently more intense in the c2/c3/c4 mutant mice, indicating a general increase in NFM production. (See FIGS. 1 and 9)

The central projections of sensory neurons also appeared defective in the mutants. Normally, these axons bifurcate into longitudinal tracts upon reaching the dorsal edge of the hindbrain or the spinal cord and course alongside the gray matter (FIGS. 1C and 1E). In the c2/c3/c4 triple mutants, in contrast, the central branches of spinal sensory neurons from the DRG failed to project longitudinally upon reaching the dorsal spinal cord at the dorsal root entry zone (FIG. 1C). As a result, the longitudinal tract or dorsal funiculus (DF) in wild-type and control littermates was well developed at E10.5 (FIG. 1E), but in Theiler stage-matched triple mutants it was absent or very fragmented (FIGS. 1F and 9D). Similarly, the central projections of trigeminal neurons also appeared defective.

The defects in the c2/c3/c4 triple mutants did not appear to be related to a failure of sensory neuron differentiation, because expression of several markers of cell type specification (βIII-Tubulin, Nkx2.2, HNF3β, Lim1/2, Pax7, Islet-1, neurogenin-1 and 2, Neuro-D, and SCG-10) was similar to that observed in littermate controls (FIG. 10). Indeed, the neurotrophin receptor genes TrkB and C were, if anything, overexpressed in c2/c3/c4 mutants. The normal expression of most differentiation markers strongly argues against a developmental delay in the NFATc mutant mice (FIG. 12).

Triple mutant embryos also displayed profound disturbances in commissural axon growth as visualized by TAG-1 staining. At E10.5, in control embryos, commissural axons project toward the floor plate and some have already crossed the midline (arrowhead and open arrowhead in FIG. 11). In contrast, very few TAG-1 positive neurites can be seen in the mutant and most of them are much shorter as they project only midway in the spinal cord (arrowhead FIG. 1J) and no TAG-1 positive axons reach the floor plate and cross the midline (open arrowhead 1J). Many NFM positive processes in the mutant were oriented along a medio-lateral trajectory (FIG. 1H) and a few axons reached the floor plate. Since these processes are TAG-1 negative, they are unlikely to be misprojections from commissural neurons or motoneurons. Instead they might represent interneurons that have failed to migrate to their proper locations or that are projecting abnormally. Again, neurons in the mutant stain more intensely for NFM.

### Example 2

Transient Calcineurin Inhibition During Embryonic Development Mimics Sensory Neuronal Defects Seen in NFA Tc2/c3/c4 Mutant Mice.

Previously characterized functions of the four NFATc genes are known to be regulated by the Ca²⁺ activated phosphatase calcineurin, which regulates their nuclear import (Clipstone and Crabtree, 1992; Klee et al., 1998). We therefore examined whether defects seen in triple mutant mice were due to a failure of transmission of a Ca²⁺/calcineurin signal to the nucleus. We found that calcineurin B mutant mice have defects in axonal outgrowth but die at E10.0 due to a failure to properly pattern the developing vascular system (Graef et al., 2001a) and data not shown). To circumvent this problem and study the role of calcineurin in axon outgrowth in embryos at later stages, we used the calcineurin inhibitor cyclosporin (CsA). CsA is a natural microbial product that crosses the placenta and binds to cyclophilin A, producing inhibitory complexes that block calcineurin phosphatase activity (Liu et al., 1991). Another chemically distinct inhibitor of calcineurin is FK506 (used in later experiments), which binds FKBP12, producing inhibitory complexes (Liu et al., 1991). The exquisite specificity of CsA or FK506 for calcineurin is based on the large and evolutionarily highly perfected composite surface used to bind the calcineurin A/B complex by the CsA/cyclophilin or FK506/FKBP complex (Griffith et al., 1995; Kissinger et al., 1995). Defects observed in E10.5 wild-type embryos treated in utero by administering CsA to the pregnant mothers were indistinguishable from those in NFATc2/c3/c4 triple mutant embryos (FIG. 2), including a profound impairment of peripheral projections of trigeminal sensory neurons (FIGS. 2A-D) and spinal sensory neurons in the DRGs (FIGS. 2D, F). In addition, defects of the central branches of DRG neurons and in specific motor neurons were seen in CsA-treated and c2/c3/c4 triple mutants (data not shown). These observations, together with earlier work (Graef et al., 2001a) indicate that at early stages of embryogenesis calcineurin may be largely dedicated to regulating NFATc function.

If calcineurin were regulating NFATc proteins in growing sensory neurons, we would expect NFATc proteins to be expressed and dephosphorylated in embryonic sensory ganglia. Indeed, NFATc4 was present and dephosphorylated in the trigeminal ganglia, DRGs, cortex and spinal cord of E13.5 embryos, but was almost undetectable in the liver and other tissues (FIG. 2G). In the heart, NFATc4 was partially dephosphorylated, consistent with the critical role of NFAT signaling in the development of the cardiovascular system (de la Pompa et al., 1998; Graef et al., 2001a). CsA does have access to the developing embryo since treatment of mothers (E7.5-E8.5) results in complete conversion of NFATc4 to the fully phosphorylated form in the embryo (FIG. 2G).

### Example 3

NFATc is Required Specifically for Neurotrophin-Dependent, but not for Neurotrophin-Independent Neurite Outgrowth.

The defects seen in the c2/c3/c4 mutant mice and the CsA-treated mice could be due either to a defect in production of cues for axon extension by pathway or target cells, or to an impairment of the axons' ability to respond to such stimuli—or both. To test for a cell-autonomous defect, we examined whether the in vivo defects in axon outgrowth were also observed in vitro when the neurons were isolated from their normal environment. We focused on trigeminal ganglia because they are among the first sensory ganglia to form, and are well developed at E10.5 when the triple mutant embryos are still alive.

Normally, axons from E10.5 trigeminal ganglia are stimulated to extend into a collagen matrix by NGF and NT3, creating a broad axon halo after 48 hrs (FIG. 3A). In contrast, little outgrowth in collagen was observed from trigeminal ganglia from c2/c3/c4 triple mutants (12.3%+/−1.3% of control explant length), or when wild-type ganglia were cultured in collagen with CsA and FK506 (13.5%+/−1.2% of control explant length), despite the presence of the same neurotrophins in these cultures (FIGS. 3B-C).

To further test for a non-neuronal contribution to the in vitro outgrowth defect, we cultured dissociated trigeminal neurons at low density on a two-dimensional laminin substrate. When dissociated trigeminal neurons from c2/c3/c4 mutant E10.5 embryos were cultured on laminin in the presence of NGF and NT3, shorter (9.9%+/−4.2% of control axon length), and fewer axons extended compared to littermate controls (FIGS. 3E and 3H). Calcineurin was also necessary for outgrowth under these culture conditions, because treatment of wild-type trigeminal neurons with FK506 and CsA (10.2%+/−4.7% of control axon length), mimicked the outgrowth defects seen in the c2/c3/c4 mutant neurons (FIGS. 3F and 3I). Together, these experiments show that loss of NFATc2/c3/c4 gene function, or inhibition of calcineurin, can impair axon outgrowth when neurons are cultured in vitro. This is observed even in low density cultures where it is difficult to argue for effects via non-neuronal cells, strongly implying that at least some of the defects observed in vivo are cell-autonomous.

Pharmacological inhibition of axon outgrowth in explant and dissociated cultures described above required use of both CsA and FK506 and only partial block was observed with either alone (data not shown). This contrasts with the ability of CsA by itself to inhibit sensory axon growth over longer periods in vivo (FIG. 2) This difference might be explained by the observation that long-term blockage of NFAT signaling suppresses expression of calcineurin and NFATc4 in our studies (FIG. 2E). while short term treatment does not. Hence complete inhibition of calcineurin in neurons at E10.5 in vitro might require both drugs to form enough inhibitory complexes to neutralize calcineurin, while prolonged treatment in vivo requires only CsA treatment.

Axon outgrowth in the in vitro assays just described and outgrowth of the peripheral branches of sensory axons in vivo are dependent on neurotrophins (Kaplan and Miller, 2000; O'Connor and Tessier-Lavigne, 1999). This raised the question whether calcineurin/NFAT signaling is required for outgrowth stimulated by neurotrophins. We took advantage of our previous observation that embryonic sensory axons will extend profusely in the absence of neurotrophins if they are grown in matrigel (FIGS. 3J and 3M), a basement membrane extract that contains several extracellular matrix proteins. Strikingly, extension of axons from c2/c3/c4 mutant trigeminal ganglia on this substrate was normal (FIG. 3 K). Similarly, inhibition of calcineurin did not affect trigeminal axon outgrowth on this substrate (FIG. 3L). The difference between outgrowth on matrigel compared to either collagen or laminin is that outgrowth on matrigel did not require neurotrophins (FIGS. 3M-O). Further, these results demonstrate that neurons from the NFATc mutant trigeminal ganglia as well as FK/CsA-treated trigeminal neurons are not generally sick or growth-arrested. These results contrast markedly with the profound impairment seen for the neurotrophin-dependent outgrowth on laminin or in collagen (FIG. 3A-I) and suggest that neurotrophins might produce their effects on axonal outgrowth in part by signaling through calcineurin and NFATc proteins.

### Example 4

NFAT Signaling is not Essential for Neurotrophin-Dependent Survival In Vivo or In Vitro

Since neurotrophins induce neurite outgrowth and promote survival during development, we determined if NFAT signaling was required for the survival effects of neurotrophins. Cell death is a normal part of CNS and PNS development, and can be observed by TUNEL staining of sections of control mice at E10.5. In the mutant mice, we did not observe a change in the number of TUNEL positive neurons in the DRGs or neural tube of mutants, and only a very slight increase in the trigeminal ganglia (FIGS. 4A-4E). Therefore, cell death is not the primary reason for the inability of axons to project to the periphery. This result is consistent with the observation that growth of mutant E10.5 trigeminal ganglia in matrigel is similar to that observed in controls (FIGS. 3K and 3N).

To further define the role of NFAT signaling on survival we used neurotrophin-dependent low-density cultures of dissociated E10.5 trigeminal neurons in serum-free medium (Buchman and Davies, 1993), in the presence or absence of NGF and NT3. Culturing the cells without neurotrophins more than doubled the amount of cell death in the cultures (FIGS. 4G and 4J). An even greater degree of cell death was induced by 150 nM Kn252a, an inhibitor of Trk kinase activity (FIGS. 4H and 4J). Thus, survival in these cultures is highly dependent on neurotrophins. In contrast, the combination of FK/CsA that completely block calcineurin activity and axon outgrowth did not increase cell death (FIGS. 4I and 4J). These data suggest that, while neurotrophin signaling under these culture conditions is essential for neuronal survival, calcineurin signaling is dispensable.

If calcineurin were not required for survival one would predict that the effects of FK/CsA inhibition of calcineurin should be fully reversible. To test this prediction, trigeminal ganglia cultured on collagen were treated with FK/CsA for 24 hours and then the drug removed by washout. Treatment of trigeminal ganglia for 48 hours with FK/CsA completely blocked axonal outgrowth (FIG. 4M). However, when the drugs were washed out after 24 hours, axonal outgrowth recovered fully (compare FIGS. 4M and 4N) after a further 48 hrs. In contrast, trigeminal ganglia cultured for 48 hours in the absence of neurotrophins showed near complete lack of outgrowth (FIG. 4L). Thus, FK/CsA treatment and block of calcineurin/FAT signaling for 24 hours does not lead to significant cell death or irreversible toxicity. These results indicate that calcineurin/NFAT signaling is selectively required for neurotrophin-dependent axon outgrowth but not neurotrophin-dependent survival.

### Example 5

Delayed but Specific Effects of Calcineurin Inhibition

A loss of NFATc function most likely results in impaired axon outgrowth because of failure to transcribe genes essential for neurite outgrowth and axon extension. However, pharmacological inhibition of calcineurin with FK/CsA could potentially impair axon outgrowth either by affecting NFAT-dependent transcription, or through a direct effect on the axons or growth cones. To test for a direct effect on axons, we first examined whether the failure of axonal outgrowth on laminin with FK/CsA reflects growth cone collapse or retraction. When cultures of trigeminal neurons were treated with FK/CsA for 16 hrs only a few growth cones formed and elongation was absent (FIG. 5B). Addition of FK/CsA for 30 minutes did not induce collapse of extending growth cones (FIG. 5C), in contrast to treatment with Sema3A (FIG. 5D). Thus, FK/CsA did not produce an immediate collapsing effect on the cytoskeleton. The fact that NFAT/calcineurin signaling was not required for axon outgrowth on matrigel allowed us to test for a role of this signaling pathway in acute semaphorin responses. Sema3A also induced efficient collapse of growth cones in cultures of trigeminal ganglia from c2/c3/c4 triple mutants or of wild-type trigeminal ganglia cultured with CsA and FK506 on matrigel (FIG. 11), demonstrating that only selective signal transduction pathways are affected by lack of calcineurin/NFATc signaling.

Additional evidence for a transcriptional role of calcineurin came from experiments in which we determined the lag-time between addition of FK/CsA and the arrest of axon outgrowth. We grew wild-type trigeminal ganglia for 24 hr in collagen gels, then added FK/CsA and followed the further growth (Δ) of the axons at the indicated times (t+24 hrs) (FIG. 5E). Quantitative analysis showed that after drug addition axonal elongation proceeded for five hours at the same rate as that observed with the non-treated explants, but at that point outgrowth slowed by a factor of 3.5 in the drug-treated explants (FIG. 5E). The delay in the onset of action of FK/CsA, and the lack of acute collapse-inducing activity, argue against a direct effect of calcineurin inhibition on axon elongation, and is consistent with a model in which the inhibitory effects of calcineurin on neurite outgrowth are transcriptional.

### Example 6

NFATc Functions Downstream of Neurotrophins

The finding that calcineurin/NFAT signaling was required for the neurotrophin-induced outgrowth but not the neurotrophin-independent outgrowth of E10.5 trigeminal axons led us to examine whether NFAT signaling is activated by neurotrophins. We tested this possibility using cultured E15.5 cortical neurons, because, unlike sensory neurons, they are not dependent on neurotrophins for their survival in culture, yet they express TrkB receptors on their surface. We found that BDNF treatment induces nuclear translocation of EGFP-tagged NFATc4 within 30 min as reflected by the disappearance of the clear nucleus in BDNF treated cells (FIG. 6A). NGF did not induce translocation, consistent with lack of expression of TrkA (data not shown). However, when EGFP-NFATc4 and TrkA were introduced into the cells by co-transfection, NGF led to rapid translocation of EGFP-NFATc4 into the nucleus (FIG. 6A). Addition of CsA and FK506 to the cultures blocked translocation (FIG. 6A). These results demonstrate that neurotrophins act directly in neurons to induce calcineurin activity and regulate NFATc4 nuclear localization.

Translocation of NFATc proteins to the nucleus is one of two stimuli that are required for activation of NFAT transcription complexes. The second stimulus usually requires ras or protein kinase C (PKC) activation (Crabtree, 1989). Since neurotrophins can activate ras and PKC, it seemed possible that they might provide the two stimuli necessary for NFAT-dependent transcription. We found that BDNF was a powerful activator of NFAT-dependent transcription in E15.5 cultured cortical neurons (FIG. 6B). BDNF-induced, NFAT-dependent transcription was blocked by FK/CsA (FIG. 6B) at concentrations that did not inhibit the expression of a constitutively active luciferase reporter gene (data not shown). NGF did not activate transcription from this reporter unless TrkA was introduced into the cells by transfection (FIG. 6C). Thus, neurotrophins can stimulate NFATc nuclear translocation and activation of NFAT-dependent transcription in cortical neurons, demonstrating a direct action of neurotrophins on NFAT-dependent transcription.

TrkA receptors transfected into cortical neurons, which lack endogenous TrkA receptors, required the PLCγ1 interaction site (Y794) or the Shc-interaction site (Y499) to activate NFAT-dependent transcription in response to NGF. The requirement for the PLCγ1 interaction site may relate to PLCγ's ability to stimulate Ca²⁺ release and the fact that Ca²⁺ is essential to activate calcineurin and induce translocation of the cytosolic subunits of NFATc transcription complexes. A requirement for the Shc-interaction site might reflect the requirement for ras/MAPK or PI3K activation for inducing the nuclear components of NFATc transcription complexes, which are PKC/ras-dependent. As a control, activation of an AP-1 reporter, which is dependent on Ras-signaling, was not affected by mutation of the PLCγ1-interaction site on TrkA, but was blocked by the Shc-interaction site mutation (data not shown).

### Example 7

Calcineurin Inhibition also Impairs Netrin-Dependent Axon Outgrowth

The defects seen in the NFATc null mice and in the CsA-treated embryos were more extensive than those expected if calcineurin and NFATc were only required for neurotrophin signaling. For example, extension defects of commissural axons in c2/c3/c4 null mice are similar to defects found in mice mutant in netrin-1 or its receptor, DCC (Fazeli et al., 1997; Serafini et al., 1996) (FIGS. IF, H). We found that the calcineurin inhibitors FK/CsA blocked the rapid (19 hours) netrin-induced axon extension from E13 rat dorsal spinal cord explants in collagen and matrigel three-dimensional cultures (FIGS. 7B, D). However, netrin-independent outgrowth (Keino-Masu et al., 1996), which is very slow and can be measured at 43 hrs was not blocked by FK/CsA (FIGS. 7A, C). These data indicate that the observed inhibitory effect does not represent a general inhibition of outgrowth, but rather inhibition of outgrowth stimulated by netrin/DCC signaling (also see Discussion of Results from Example).

We found that netrin activated endogenous NFAT-dependent transcription by about 2- to 3-fold in E15.5 cortical neurons (FIG. 7E). This increase appeared to be calcineurin-dependent since it was blocked by FK/CsA. Because cortical neurons may not have saturating levels of the netrin receptor DCC, we co-transfected DCC with the reporter construct and found that netrin induced about a 4- to 5-fold increase in NFATc activity, suggesting that DCC was limiting in E15.5 cortical neurons. To further determine if transcription was dependent on netrin, we cotransfected a dominant-negative version of DCC lacking its cytoplasmic domain (Dn DCC) and found that it blocked NFAT-dependent transcription (FIG. 7E). These observations indicate that netrin is a powerful activator of endogenous NFAT-dependent transcription in cultured cortical neurons.

Together, these observations suggest that the defects in commissural axon outgrowth observed in vivo in NFATc triple-mutant embryos could be due partly or even entirely to loss of NFATc function in neurons. We cannot yet fully exclude that the failure of commissural axon growth could in part reflect a defect in presentation of cues in the environment; however, we have found that expression of netrin-1 and DCC mRNAs were normal in E10.5 triple mutants.

### Example 8

Down-Regulation of NFATc4 Expression in Adult Neurons

FIG. 13 is a Western blot showing the downregulation of NFATc4 in adult neurons.

This observation is consistent with the reduction of outgrowth and regeneration capabilities of adult CNS neurons. This result suggests that postnatal repression of NFATc4 contributes to the inability of adult CNS neurons to regenerate, and suggests that NFATc can be used to induce regeneration of adult CNS neurons.

### Example 9

NFATc2, c3 and c4 are Induced in DRGs After Transection of the Sciatic Nerve (“Axotomony”).

The Examples above show that the disruption of Ca²⁺/calcineurin/NFAT signaling leads to defects in embryonic axon outgrowth. These data indicate that induction of a transcriptional program of axonal outgrowth requires signaling by Ca²⁺, calcineurin and NFAT. It has been shown that peripheral—but not central—axotomy induces a transcription-dependent change that alters the type of axon growth that can be executed by adult dorsal root ganglia (DRG) neurons and thus a change in their regenerative capacity (Smith, D. S. and J. H. Skene, A transcription-dependent switch controls competence of adult neurons for distinct modes of axon growth. Journal of Neuroscience, 1997. 17(2): p. 646-658). Thus, genes that are induced following peripheral axotomy are thought to increase the intrinsic growth capacity of adult neurons.

Hence, we investigated whether NFATc genes, which are required for embryonic axon outgrowth, are transcriptionally induced following peripheral axotomy. We tested whether NFATc RNA expression in L4/L5 DRGs increases after transaction of the sciatic nerve of wild type mice. The contralateral side of the animal served as a control in all of these experiments. Using RT-PCR we found that the RNA expression of NFATc2, c3 and c4 increased 24 hours after peripheral but not central axotomny (FIG. 14).

### Example 10

NFATc2^{-/-}, c3^{+/-}, c4^{-/-} Mutant Mice Showed Defects in Activation of an “Elongating” Growth Program After Sciatic Nerve Transaction.

Since NFATc2, c3 and c4 are essential for growth factor induced rapid axonal extension during embryonic development and are induced after peripheral but not after central axotomy we proceeded to test if NFATc2^{-/-},c3^{+/-},c4^{-/-} mice showed defects in activation of an “elongating” growth program after sciatic nerve transection (axotomy). Neurons subjected to peripheral axotomy before plating support a distinct mode of growth characterized by rapid extension of axons. L4/L5 DRGs from NFATc2^{-/-},c3^{+/-},c4^{-/-} mutant mice and control animals were removed 3 days after sciatic nerve transection, dissociated and plated on laminin. 24 hours after plating we could observe a small, but significant reduction in axon outgrowth of mutant DRG neurons (FIG. 15).

### Example 11

Endogenous NFATc4 Interacts with Endogenous Brg-1 Protein in Primary Embryonic Cortical Neurons.

Chromatin modification and remodeling are the principle epigenetic mechanisms cells use to establish and maintain their specific gene expression patterns during development. Changes in chromatin architecture could allow the same transcription factors to activate distinct sets of genes at different developmental stages. Consequently, a signaling pathway and/or transcription factor that would be capable of reprogramming adult neuron and reintroduce specific embryonic outgrowth programs, is likely to modify not only transcription but also the chromatin structure of critical target genes. A variety of chromatin remodeling complexes are thought to assist sequence-specific transcription factors. ATP-dependent chromatin remodeling complexes use energy derived from ATP hydrolysis to overcome repressive chromatin structures, change its accessibility and regulate gene expression (Olave, I. A., S. L. Reck-Peterson, and G. R. Crabtree, Nuclear actin and actin-related proteins in chromatin remodeling. Annu Rev Biochem, 2002. 71: p. 755-81). The first such complex, SWI/SNF was identified in yeast for its roles in mating type switching and sucrose fermentation in response to external signals. Related complexes containing a SWI2/SNF2 homolog as their core ATPase subunit were purified from other organisms. Mammalian cells contain two such ATPases, hBrm and Brg (Khavari, P. A., et al., BRG1 contains a conserved domain of the SWI2/SNF2 family necessary for normal mitotic growth and transcription. Nature, 1993. 366(6451): p. 170-4). Using antibodies against Brg, a family of complexes related to the yeast SWI/SNF.complex was purified and termed BAF (Brg Associated Factors) (Wang, W., et al., Purification and biochemical heterogeneity of the mammalian SWI-SNF complex. Embo J, 1996. 15(19): p. 5370-82; Wang, W., et al., Diversity and specialization of mammalian SWI/SNF complexes. Genes Dev, 1996. 10(17): p. 2117-30). Recently it was reported that murine neurons have a specific chromatin remodeling complex (bBAF) based on the neuron specific expression of BAF53b (Olave, I., et al., Identification of a polymorphic, neuron-specific chromatin remodeling complex. Genes Dev, 2002. 16(19): p. 2509-17). When we tested whether NFATc proteins can interact with chromatin modifying proteins, we found that endogenous NFATc4 tightly interacts with endogenous Brg-1 protein in primary embryonic cortical neurons (FIG. 16). Brg-1 co-immunoprecipitated with NFATc4 and vice versa, while neither protein interacted with endogenous β-catenin, engrailed, Neu-N under these conditions (FIG. 16 and data not shown). Upon reintroduction of NFATc4 into adult neurons, the BAF-complex might be recruited to gene loci that are repressed in adult neurons. Changing the chromatin architecture of these loci might then allow the re-expression of embryonic genes that are critical for rapid axon elongation.

### Example 12

Miscellaneous Protocols

a. Generation of NFATc2/c3/c4 Triple Knock-Out Mice and CsA Treatment of Embryos

Triple knock-out mice were generated by intercrossing of NFATc3/c4 double mutant mice (Graef et al., 2001b) with NFATc2 mutant mice (Hodge et al., 1996). CsA treatment of time-pregnant females was performed as previously described (Graef et al., 2001b).

b. Immunohistochemistry and Tunel Assays

Embryos were obtained from timed pregnancies with the noon of the plug date defined as E10.5. For whole-mount studies, embryos were processed and stained with anti-Neurofilament antibody (clone 2H3, DSHB) and processed for DAB or fluorescent staining. Sections were stained with anti-NF-M antibody, anti-TAG-1 antibody, anti-Lim1/2, anti-Pax7, anti-Nkx2.2, anti HNF-3b, anti-islet-1 (all from DSHB), Tuj-1 (ABR) and anti-TrkC (Chemicon). Secondary antibodies used were HRP conjugated goat anti-mouse antibody (Jackson Immunoresearch), Alexa-594 and Alexa-488 conjugated goat anti-mouse antibody (Molecular Probes) as well as anti-mouse and anti-rabbit ABC kits (Vector). Tunel assays were performed according to the manufacturer's recommendations (Roche).

c. Explant Cultures and in Vitro Assays.

E10.5 trigeminal explants were cultured in either collagen or matrigel (Becton-Dickinson) gels in the presence of NT-3 (Gibco) and NGF (Gibco) as previously described (O'Connor and Tessier-Lavigne, 1999). The explants were then fixed in 4% PFA/PBS and axons were visualized by wholemount immunostaining with the anti-NF-M antibody (O'Connor and Tessier-Lavigne, 1999). For cultures on laminin, eight-well chamber tissue culture slides were coated with poly-L-lysine followed by laminin. Trigeminal ganglia were either dissociated or cut into small cubes and cultured in medium supplemented with NT-3 and NGF. For assessing growth cone morphology cells were fixed with 25% sucrose in 4% PFA/PBS and stained with either Alexa 594-phalloidin (Molecular Probes) alone or plus anti-III β-tubulin antibody (Tuj-1, Covance).

d. Cell Culture and Transfections

E14.5 mouse and E16.5 rat cortical neurons were cultured as previously described (Tao et al., 1998) and transfected after 3 days of in vitro culture as described previously. The reporter plasmid NFAT-Luciferase and the EGFP-NFATc4 plasmid have been described elsewhere (Graef et al., 1999). The TrkA constructs have been previously described (Ming et al., 1999). For luciferase assays the cells were treated 16 hrs after transfection with 100 ng/ml recombinant BDNF (Gibco) or 100 ng/ml recombinant NGF. For studies with FK506/CsA the cells were preincubated for 10 min with the inhibitors before stimulation and the inhibitors were present during the stimulation. Cells were lysed for luciferase assays 18 hrs after stimulation and luciferase assays performed according to standard protocols. For microscopy, neurons were fixed in 4% formaldehyde/PBS. Co-transfected m yc-epitope tagged Trk-A was visualized by staining with anti-myc antibody (clone 4E10, Pharmingen), followed by Alexa-594 conjugated goat anti-mouse antibody (Molecular Probes).

e. Survival Assay

Trigeminal ganglia were incubated for 5 minutes at 37° C. with 10 mg/ml trypsin (Sigma)+DNAse (Sigma) in calcium- and magnesium-free (CMF) HBSS. After removal of the trypsin solution, the ganglia were washed 3 times with Hams F12 medium containing 10% heat-inactivated horse serum washed 3 times with CMF-HBSS and were gently triturated in 1 ml CMF-HBSS+DNAse with a fire-polished, siliconised Pasteur pipettes to give a single cell suspension. The cells were plated at a density of 2500 neurons per well onto 8-well tissue-culture slides that had been precoated with poly-L-Lysine (Sigma, 0.5 mg/ml, 1 hour at room temperature) and laminin (Gibco, 20 mg/ml for 4 hours at 37° C). Neurons were cultured in serum-free medium in either the presence or absence of NT-3 and NGF and at the onset of culture either 100 ng/ml of FK506, 1 μg/ml of CsA or 150 nM Kn252a were added to the medium.

f. Imaging

Anti-NF-M stained embryos and explants were photographed using a Leica DC 500 camera. Fluorescent stain whole-mount embryos and trigeminal explants were scanned using a two-photon microscope (Zeiss LSM510 with a Coherent MIRA laser, Stanford Imaging Facility). Images for Tunel staining were collected with a deconvolution microscope (DeltaVision, at Stanford Cell Imaging Facility).

g. Dorsal Spinal Cord Explant Cultures

Explants of E13 rat dorsal spinal cord were isolated and cultured as described (Serafini et al., 1994). For explants cultured in matrigel, a 1:3 matrigel:collagen dilution was used. Netrin-dependent outgrowth of commissural axons was elicited by a ddition of 100 ng/ml of purified netrin-1 and was assessed after 19 hours. For netrin-independent outgrowth assays dorsal spinal cord explants were cultured for 43 hours in the absence of netrin. Increasing concentrations of FK506 and CsA were added to the culture media during the first hour of explant culture. Dorsal spinal cord explants were stained by whole-mount immunohistochemistry with the anti-NF-M antibody as described. A measure of total axon bundle length per explant was obtained by adding the lengths of all axons from each explant.

Discussion of Results from Examples

We have presented several lines of evidence that signaling through calcineurin and NFATc proteins play critical roles in regulating embryonic axon outgrowth from a variety of neuronal classes. Based on these data we propose that embryonic axon outgrowth stimulated by growth factors such as neurotrophins and netrins requires these factors not only to stimulate the tips of growth cones, but also to selectively activate a calcineurin/NFAT-dependent transcriptional program controlling the rate of axonal extension.

NFAT Signaling Functions in Neurons to Promote Embryonic Axon Growth.

The dramatic defects in nervous system development in NFATc2/c3/c4 triple mutants and in CsA-treated embryos, do not appear to result from defects in cell specification, as assessed by the normal expression of a variety of markers of neuronal identity. The defects also do not reflect a major increase in cell death, because we did not observe enhanced apoptosis in mutant embryos. Furthermore, inhibition of calcineurin/NFAT signaling with FK/CsA reversibly blocks sensory axon growth from explants in collagen, and does not increase sensory neuron death in low-density cultures. The idea that the primary defect is a defect in axon growth is further supported by the appearance of mutant sensory ganglia, which have an apparently normal shape despite the short length of axons.

In principle, the growth defects in vivo in NFATc triple mutants could result from a defect in the neurons, a defect in the environment through which their axons must grow, or both. Our in vitro data strongly suggest that the in vivo phenotypes reflect at least partly a defect in the neurons, since outgrowth into collagen of trigeminal or commissural axons from explants in response to neurotrophins or netrins is inhibited by pharmacological (and in the case of trigeminal ganglia genetic) blockade of NFAT signaling. These in vitro cultures are believed to be representative models of growth of these axons in their normal environments. These results are consistent with calcineurin/NFAT signaling being required in the neurons themselves, although they do not establish this point conclusively, since those explants contain non-neuronal cells as well. More conclusive evidence for a cell-autonomous requirement for NFAT signaling in axon growth is, however, provided in the case of sensory axons by axon outgrowth defects in low-density cultures, where invoking indirect effects via non-neuronal cells is even less plausible. While these results thus support a cell-autonomous requirement for calcineurin/NFAT signaling in outgrowth of these axons in vivo, we currently cannot exclude an additional role for NFAT signaling in surrounding or supporting cells. In preliminary studies, however, we have not found a change in expression of neurotrophins, netrins or their receptors in the triple mutants. Further studies will be required to determine whether environmental defects contribute in any way to the axon outgrowth defects seen in vivo.

How does NFATc participate in cell-autonomous regulation of axon growth?

At one extreme, NFATc proteins could perform a general function in neurons that simply affects axon growth in some indirect manner. However, the following lines of evidence indicate a more central role for NFATc proteins in the sustained transduction of signals for axon growth downstream of growth factors like neurotrophins and netrins.

1.) The phenotype of NFATc triple mutant mice can be mimicked by in vivo inhibition of calcineurin through administration of CsA to embryos, indicating that calcineurin-regulated NFATc activity is specifically important for the in vivo phenotype. The same is true in vitro, where outgrowth defects of triple mutant trigeminal ganglia are reproduced by pharmacological calcineurin inhibition of wild-type ganglia. The reversibility of the pharmacological inhibition, and the ability to shut down outgrowth with late addition of FK/CsA, demonstrate that ongoing stimulation of calcineurin/NFAT signaling is required for axon outgrowth in vitro and in vivo.

2.) Neurotrophins and netrins directly activate NFAT transcriptional activity in vitro, presumably by inducing NFATc4 nuclear translocation (as demonstrated directly for neurotrophins but not yet netrins). Intracellular Ca²⁺ transients elicited by neurotrophins and netrins have an important role in regulating growth cone motility and axon growth (Hong et al., 2000; Lankford and Letourneau, 1989; Ming et al., 2002), and might also underlie the activation of calcineurin/NFAT-dependent transcription by these factors.

3.) NFAT signaling is required for growth factor-dependent, but not growth factor-independent extension of sensory and commissural axons. This idea is most strongly supported in the case of trigeminal sensory neurons, since genetic and pharmacological blockade of calcineurin/NFAT signaling both impair neurotrophin-dependent growth of these axons in collagen or on laminin, but not their neurotrophin-independent growth in matrigel. Clear, though less complete, evidence for this idea was also obtained in the case of commissural neurons, since NFAT-signaling is required for the netrin-dependent but not the late netrin-independent extension of commissural axons in collagen.

Taken together, these results support a model in which neurotrophins and netrins, in addition to their direct actions on growth cone tips, must activate a calcineurin-NFAT-dependent transcriptional program that is required in an ongoing way for efficient embryonic axon outgrowth in response to these factors.

The impairment of trigeminal axon growth in vivo in NFATc triple mutants is greater than might be expected from complete loss of neurotrophin signaling, and the impairment of commissural axon growth in these animals is also more severe than in either netrin-1 or DCC mutant embryos. These observations suggest that other growth factors collaborate with neurotrophins and netrins to stimulate the extension of trigeminal and commissural axon growth in vivo, and that these factors also must activate NFAT signaling to produce their effects. These considerations raise the possibility that activation of calcineurin/NFAT signaling might be required quite generally for stimulation of embryonic axon outgrowth by growth factors.

Independent Control of Axonal Extension and Survival: a Rationale for the Selectivity of NFAT Signaling

Although sensory neurons lacking NFAT signaling are unable to extend axons efficiently in response to neurotrophins they do not appear to be compromised in their ability to interpret the survival promoting activity of neurotrophins. Indeed, in vivo the dramatic defects in axon extension seen in NFATc2/c3/c4 mutants is not accompanied by a dramatic increase in cell death. This precise parsing of signals for survival and for axon extension could allow independent control of these two processes by factors encountered along the paths of axons to their targets, and independent regulation of these two effects for a given factor. Independent control of these two processes is in fact observed. For example, embryonic sensory axons initially respond to neurotrophins with rapid axon outgrowth, but when they reach their targets they stop extending rapidly in response to these factors (instead responding by elaborating their terminal arbors) at the same time as they actually become more dependent on neurotrophins for their survival. A switching off of the calcineurin/NFAT signaling pathway could in principle underlie the switch from an elongating to an arborizing mode in these neurons, without affecting their trophic dependence on neurotrophins.

Our data thus define a dedicated signaling and transcriptional program required for growth-factor stimulated axon outgrowth of embryonic axons. The finding of such a program was surprising, as we believe it has been implicitly assumed that the ability of embryonic neurons to extend an axon in response to growth-stimulating factors is simply another generic aspect of an intrinsic neuronal specification program. This implicit assumption was perhaps reinforced by the evidence that growth factors like neurotrophins stimulate axon extension by acting on growth cones at the tips of axons, far from their cell bodies (e.g. (Campenot, 1977)). Our results indicate that, while likely necessary, stimulation of axon tips is not apparently sufficient for sustaining the rapid growth induced by the growth factors, and that sustained activation of NFAT-dependent transcription is also required.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

### References

Anderson, D. J. (2001). Stem cells and pattern formation in the nervous system: the possible versus the actual. Neuron 30, 19-35.

Arber, S., Ladle, D. R., Lin, J. H., Frank, E., and Jessell, T. M. (2000). ETS gene Er81 controls the formation of functional connections between group Ia sensory afferents and motor neurons. Cell 101, 485-498.

Beals, C. R., Clipstone, N. A., Ho, S. N., and Crabtree, G. R. (1997a). Nuclear localization of NF-ATc by a calcineurin-dependent, cyclosporin- sensitive Intramolecular interaction. Genes Dev 11, 824-834.

Beals, C. R., Sheridan, C. M., Turck, C. W., Gardner, P., and Crabtree, G. R. (1997b). Nuclear export of NF-ATc enhanced by glycogen synthase kinase-3. Science 275, 1930-1934.

Bibel, M., and Barde, Y. A. (2000). Neurotrophins: key regulators of cell fate and cell shape in the vertebrate nervous system. Genes Dev 14, 2919-2937.

Brenman, J. E., Gao, F. B., Jan, L. Y., and Jan, Y. N. (2001). Sequoia, a tramtrack-related zinc finger protein, functions as a pan-neural regulator for dendrite and axon morphogenesis in Drosophila. Dev Cell 1, 667-677.

Briscoe, J., Pierani, A., Jessell, T. M., and Ericson, J. (2000). A homeodomain protein code specifies progenitor cell identity and neuronal fate in the ventral neural tube. Cell 101, 435-445.

Buchman, V. L., and Davies, A. M. (1993). Different neurotrophins are expressed and act in a developmental sequence to promote the survival of embryonic sensory neurons. Development 118, 989-1001.

Casadio, A., Martin, K. C., Giustetto, M., Zhu, H., Chen, M., Bartsch, D., Bailey, C. H., and Kandel, E. R. (1999). A transient, neuron-wide form of CREB-mediated long-term facilitation can be stabilized at specific synapses by local protein synthesis. Cell 99, 221-237.

Clipstone, N. A., and Crabtree, G. R. (1992). Identification of calcineurin as a key signalling enzyme in T cell activation. Nature 357, 695-697.

Condron, B. G. (1999). Serotonergic neurons transiently require a midline-derived FGF signal. Neuron 24, 531-540.

Crabtree, G. R. (1989). Contingent Genetic Regulatory Events in T Lymphocyte Activation. Science 243, 355-361.

Crabtree, G. R., and Olson, E. N. (2002). NFAT signaling: choreographing the social lives of cells. Cell 109 Suppl, S67-79.

Davies, A. M. (2000). Neurotrophins: more to NGF than just survival. Curr Biol 10, R374-376.

Deckwerth, T. L., Elliott, J. L., Knudson, C. M., Johnson, E. M., Jr., Snider, W. D., and Korsmeyer, S. J. (1996). BAX is required for neuronal death after trophic factor deprivation and during development. Neuron 17, 401-411.

Deisseroth, K., Heist, E. K., and Tsien, R. W. (1998). Translocation of calmodulin to the nucleus supports CREB phosphorylation in hippocampal neurons. Nature 392, 198-202.

Dodd, J., and Jessell, T. M. (1988). Axon guidance and the patterning of neuronal projections in vertebrates. Science 242, 692-699.

Dolmetsch, R. E., Lewis, R. S., Goodnow, C. C., and Healy, J. I. (1997). Differential activation of transcription factors induced by Ca²⁺ response amplitude and duration [published erratum appears in Nature 1997 Jul 17;388(6639):308]. Nature 386, 855-858.

Edlund, T., and Jessell, T. M. (1999). Progression from extrinsic to extrinsic signaling in cell fate specification: a view from the nervous system. Cell 96, 211-224.

Flanagan, W. M., Corthesy, B., Bram, R. J., and Crabtree, G. R. (1991). Nuclear association of a T-cell transcription factor blocked by FK-506 and cyclosporin A. Nature 352, 803-807.

Gao, F. B., Brenman, J. E., Jan, L. Y., and Jan, Y. N. (1999). Genes regulating dendritic outgrowth, branching, and routing in Drosophila. Genes Dev 13, 2549-2561.

Giger, R. J., and Kolodkin, A. L. (2001). Silencing the siren: guidance cue hierarchies at the CNS midline. Cell 105, 1-4.

Goldberg, J. L., Espinosa, J. S., Xu, Y., Davidson, N., Kovacs, G. T., and Barres, B. A. (2002). Retinal ganglion cells do not extend axons by default: promotion by neurotrophic signaling and electrical activity. Neuron 33, 689-702.

Graef, I. A., Chen, F., Chen, L., Kuo, A., and Crabtree, G. R. (2001a). Signals transduced by Ca(2+)/calcineurin and NFATc3/c4 pattern the developing vasculature. Cell 105, 863-875.

Graef, I. A., Chen, F., and Crabtree, G. R. (2001b). NFAT signaling in vertebrate development. Curr Opin Genet Dev 11, 505-512.

Graef, I. A., Mermelstein, P. G., Stankunas, K., Neilson, J. R., Deisseroth, K., Tsien, R. W., and Crabtree, G. R. (1999). L-type calcium channels and GSK-3 regulate the activity of NF-ATc4 in hippocampal neurons. Nature 401, 703-708.

Griffith, J. P., Kim, J. L., Kim, E. E., Sintchak, M. D., Thomson, J. A., Fitzgibbon, M. J., Fleming, M. A., Caron, P. R., Hsiao, K., and Navia, M. A. (1995). X-ray structure of calcineurin inhibited by the immunophilin-immunosuppressant FKBP12-FK506 complex. Cell 82, 507-522.

Hodge, M. R., Ranger, A. M., Charles de la Brousse, F., Hoey, T., Grusby, M. J., and Glimcher, L. H. (1996). Hyperproliferation and dysregulation of IL-4 expression in NF-ATp-deficient mice. Immunity 4, 397-405.

Hong, K., Nishiyama, M., Henley, J., Tessier-Lavigne, M., and Poo, M. (2000). Calcium signalling in the guidance of nerve growth by netrin-1. Nature 403, 93-98.

Huang, E. J., and Reichardt, L. F. (2001). Neurotrophins: roles in neuronal development and function. Annu Rev Neurosci 24, 677-736.

Jain, J., McCaffrey, P. G., Miner, Z., Kerppola, T. K., Lambert, J. N., Verdine, G. L., Curran, T., and Rao, A. (1993). The T-cell transcription factor NFATp is a substrate for calcineurin and interacts with Fos and Jun. Nature 365, 352-355.

Kania, A., Johnson, R. L., and Jessell, T. M. (2000). Coordinate roles for LIM homeobox genes in directing the dorsoventral trajectory of motor axons in the vertebrate limb. Cell 102, 161-173.

Kaplan, D. R., and Miller, F. D. (2000). Neurotrophin signal transduction in the nervous system. Curr Opin Neurobiol 10, 381-391.

Kater, S. B., and Mills, L. R. (1991). Regulation of growth cone behavior by calcium. J Neurosci 11, 891-899.

Kissinger, C. R., Parge, H. E., Knighton, D. R., Lewis, C. T., Pelletier, L. A., Tempczyk, A., Kalish, V. J., Tucker, K. D., Showalter, R. E., and Moomaw, E. W. (1995). Crystal structures of human calcineurin and the human FKBP12-FK506-calcineurin complex. Nature 378, 641-644.

Klee, C. B., Crouch, T. H., and Krinks, M. H. (1979). Calcineurin: a calcium- and calmodulin-binding protein of the nervous system. ProcNatlAcadSciUSA 76, 6270-6273.

Lankford, K. L., and Letoumeau, P. C. (1989). Evidence that calcium may control neurite outgrowth by regulating the stability of actin filaments. J Cell Biol 109, 1229-1243.

Lin, J. H., Saito, T., Anderson, D. J., Lance-Jones, C., Jessell, T. M., and Arber, S. (1998). Functionally related motor neuron pool and muscle sensory afferent subtypes defined by coordinate ETS gene expression. Cell 95, 393-407.

Liu, J., Farmer, J. D., Jr., Lane, W. S., Friedman, J., Weissman, I., and Schreiber, S. L. (1991). Calcineurin is a common target of cyclophilin-cyclosporin A and FKBP-FK506 complexes. Cell 66, 807-815.

Lonze, B. E., Riccio, A., Cohen, S., and Ginty, D. D. (2002). Apoptosis, axonal growth defects, and degeneration of peripheral neurons in mice lacking CREB. Neuron 34, 371-385.

Mantamadiotis, T., Lemberger, T., Bleckmann, S. C., Kern, H., Kretz, O., Martin Villalba, A., Tronche, F., Kellendonk, C., Gau, D., Kapfhammer, J., et al. (2002). Disruption of CREB function in brain leads to neurodegeneration. Nat Genet 31, 47-54.

Ming, G., Song, H., Berninger, B., Inagaki, N., Tessier-Lavigne, M., and Poo, M. (1999). Phospholipase C-gamma and phosphoinositide 3-kinase mediate cytoplasmic signaling in nerve growth cone guidance. Neuron 23, 139-148.

Ming, G. L., Song, H. J., Berninger, B., Holt, C. E., Tessier-Lavigne, M., and Poo, M. M. (1997). cAMP-dependent growth cone guidance by netrin-1. Neuron 19, 1225-1235.

Ming, G. L., Wong, S. T., Henley, J., Yuan, X. B., Song, H. J., Spitzer, N. C., and Poo, M. M. (2002). Adaptation in the chemotactic guidance of nerve growth cones. Nature 417, 411-418.

O'Connor, R., and Tessier-Lavigne, M. (1999). Identification of maxillary factor, a maxillary process-derived chemoattractant for developing trigeminal sensory axons. Neuron 24, 165-178.

Okamura, H., Aramburu, J., Garcia-Rodriguez, C., Viola, J. P., Raghavan, A., Tahiliani, M., Zhang, X., Qin, J., Hogan, P. G., and Rao, A. (2000). Concerted dephosphorylation of the transcription factor NFAT1 induces a confornational switch that regulates transcriptional activity. Mol Cell 6, 539-550.

Patapoutian, A., Backus, C., Kispert, A., and Reichardt, L. F. (1999). Regulation of neurotrophin-3 expression by epithelial-mesenchymal interactions: the role of Wnt factors. Science 283, 1180-1183.

Patel, T. D., Jackman, A., Rice, F. L., Kucera, J., and Snider, W. D. (2000). Development of sensory neurons in the absence of NGF/TrkA signaling in vivo. Neuron 25, 345-357.

Porter, C. M., Havens, M. A., and Clipstone, N. A. (2000). Identification of amino acid residues and protein kinases involved in the regulation of NFATc subcellular localization. J Biol Chem 275, 3543-3551.

Rao, Y., Pang, P., Ruan, W., Gunning, D., and Zipursky, S. L. (2000). brakeless is required for photoreceptor growth-cone targeting in Drosophila. Proc Natl Acad Sci USA 97, 5966-5971.

Schlaeger, T. M., Qin, Y., Fujiwara, Y., Magram, J., and Sato, T. N. (1995). Vascular endothelial cell lineage-specific promoter in transgenic mice. Development 121, 1089-1098.

Senti, K., Keleman, K., Eisenhaber, F., and Dickson, B. J. (2000). brakeless is required for lamina targeting of R1-R6 axons in the Drosophila visual system. Development 127, 2291-2301.

Serafini, T., Colamarino, S. A., Leonardo, E. D., Wang, H., Beddington, R., Skarnes, W. C., and Tessier-Lavigne, M. (1996). Netrin-1 is required for commissural axon guidance in the developing vertebrate nervous system. Cell 87, 1001-1014.

Serafini, T., Kennedy, T. E., Galko, M. J., Mirzayan, C., Jessell, T. M., and Tessier-Lavigne, M. (1994). The netrins define a family of axon outgrowth-promoting proteins homologous to C. elegans UNC-6. Cell 78, 409-424.

Sharma, K., Sheng, H. Z., Lettieri, K., Li, H., Karavanov, A., Potter, S., Westphal, H., and Pfaff, S. L. (1998). LIM homeodomain factors Lhx3 and Lhx4 assign subtype identities for motor neurons. Cell 95, 817-828.

Shaw, J.-P., Utz, P. J., Durand, D. B., Toole, J. J., Emmel, E. A., and Crabtree, G. R. (1988). Identification of a putative regulator of early T cell activation genes. Science 241, 202-205.

Shirasaki, R., and Pfaff, S. L. (2002). Transcriptional codes and the control of neuronal identity. Annu Rev Neurosci 25, 251-281.

Takei, K., S hin, R. M., Inoue, T., Kato, K., and M ikoshiba, K. (1998). Regulation of nerve growth mediated by inositol 1,4,5-trisphosphate receptors in growth cones. Science 282, 1705-1708.

Tao, X., Finkbeiner, S., Arnold, D. B., Shaywitz, A. J., and Greenberg, M. E. (1998). Ca2+ influx regulates BDNF transcription by a CREB family transcription factor-dependent mechanism. Neuron 20, 709-726.

Tessier-Lavigne, M., and Goodman, C. S. (1996). The molecular biology of axon guidance. Science 274, 1123-1133.

Thor, S., Andersson, S. G., Tomlinson, A., and Thomas, J. B. (1999). A LIM-homeodomain combinatorial code for motor-neuron pathway selection. Nature 397, 76-80.

Timmerman, L. A., Clipstone, N. A., Ho, S. N., Northrop, J. P., and Crabtree, G. R. (1996). Rapid shuttling of NF-AT in discrimination of Ca2+ signals and immunosuppression. Nature 383, 837-840.

Tsuchida, T., Ensini, M., Morton, S. B., Baldassare, M., Edlund, T., Jessell, T. M., and Pfaff, S. L. (1994). Topographic organization of embryonic motor neurons defined by expression of LIM homeobox genes. Cell 79, 957-970.

Von Bernhardi, R., and Bastiani, M. J. (1995). Requirement of RNA synthesis for pathfinding by growing axons. J Comp Neurol 357, 52-64.

Weimann, J. M., Zhang, Y. A., Levin, M. E., Devine, W. P., Brulet, P., and McConnell, S. K. (1999). Cortical neurons require Otxl for the refinement of exuberant axonal projections to subcortical targets. Neuron 24, 819-831.

Zhang, Y. A., Okada, A., Lew, C. H., and McConnell, S. K. (2002). Regulated nuclear trafficking of the homeodomain protein otxl in cortical neurons. Mol Cell Neurosci 19, 430-446.

Zheng, J. Q. (2000). Turning of nerve growth cones induced by localized increases in intracellular calcium ions. Nature 403, 89-93.

Zhu, J., Shibasaki, F., Price, R., Guillemot, J. C., Yano, T., Dotsch, V., Wagner, G., Ferrara, P., and McKeon, F. (1998). Intramolecular masking of nuclear import signal on NF-AT4 by casein kinase I and MEKK1. Cell 93, 851-861.

1. A method for promoting a xonal growth comprising treating a neuron with an NF-AT agonist.

2. The method of claim 1, wherein said NF-AT agonist interacts with calcineurin and increases the dephosphorylation of NF-AT.

3. The method of claim 1, wherein said NF-AT agonist binds NF-AT and increases its nuclear localization.

4. The method of claim 1, wherein the NF-AT agonist is calcineurin or an agent that upregulates the expression of calcineurin.

5. The method of claim 1, wherein the NF-AT agonist is an inhibitor of GSK-3.

6. The method of claim 5, wherein the inhibitor of GSK-3 is selected from the group consisting of an RNAi molecule, a ribozyme or a DNA enzyme that inhibits the expression of Gsk3.

7. A method for promoting axonal growth comprising treating a neuron with a composition comprising an NF-AT agonist and another agent selected from the group consisting of: a neurotrophic factor, a neuropoietic factor, inosine, a fibroblast growth factor, an insulin-like growth factor, a platelet-derive growth factor, an anti-inflammatory, anti-NGF, anti-BDNF, anti-IGF-I, transforming growth factor-beta 1, other agents that increase production of inducible-nitric oxide synthase (i-NOS), an activator of macrophages, LPS, indomethacin, and a leukemia inhibitory factor (LIF).

8. A method for promoting axonal growth comprising administering an NF-AT agonist in a biodegradable nerve conduit.

9. A method to activate NF-AT dependent gene transcription comprising the use of a netrin or a neurotrophin.

10. A method to induce regeneration of neurons comprising treating said neurons with an NF-AT agonist.

11. The method of claim 10, wherein the NF-AT agonist enhances expression of NF-AT.

12. A pharmaceutical composition comprising an NFAT agonist and a pharmaceutically acceptable carrier.

13. A method of identifying a compound that is an NF-AT agonist and promotes axonal growth comprising:
(a) contacting the compound with a cell comprising NF-AT;
(b) determining the location of NF-AT within the cell in the presence and in the absence of the compound, wherein an increase of NF-AT in the nucleus indicates that the compound is an NF-AT agonist; and
(c) determining whether the compound promotes axonal growth.

14. A method of identifying a compound that is an NF-AT agonist and promotes axonal growth comprising:
(a) contacting a cell expressing NF-AT with a compound;
(b) determining the phosphorylation state of NF-AT in the presence and absence of the compound; wherein a decrease in the phosphorylation of NF-AT indicates that the compound is an NF-AT agonist; and
(c) determining whether the compound promotes axonal growth.

15. A method of identifying a compound that is an NF-AT agonist and promotes axonal growth comprising:
(a) contacting NF-AT with a phosphatase under conditions that allow the dephosphorylation of NF-AT in the presence and in the absence of a compound,
(b) determining the phosphorylation state of NF-AT, wherein an decrease in the phosphorylation indicates that the compound is an NF-AT agonist; and
(c) determining whether the compound promotes axonal growth.

16. The method of claim 15, wherein the phosphatase is calcineurin.

17. A method of identifying a compound that is an NF-AT agonist and promotes axonal growth comprising:
(a) contacting NF-AT with a kinase under conditions that allow the phosphorylation of NF-AT in the presence and in the absence of a compound,
(b) determining the phosphorylation state of NF-AT, wherein an decrease in the phosphorylation indicates that the compound is an NF-AT agonist; and
(c) determining whether the compound promotes axonal growth.

18. The method of claim 17 wherein the kinase is GSK-3.

19. A method of determining whether a compound is an NF-AT agonist comprising:
(a) transfecting a cell with an expression vector comprising a nucleic acid encoding a reporter gene operatively linked to an NF-AT dependent transcriptional regulatory sequence;
(b) incubating the cell in the presence and absence of a compound;
(c) measuring the expression of the reporter gene; wherein an increase in the expression of the reporter gene indicates that the compound is an NF-AT agonist; and
(d) determining whether the compound promotes axonal growth.
